SEQUENCE LISTING (19 sequences)

SEQ ID NO: 1 - DNA, 1000 bp, Artificial Sequence
  misc_feature
ggctagcagggctcttcccaccacacgtttaaagcgcagtgcgaaccatgattccagcctaagaaaggcgtggccgaacataaggataagtcaaaagcgtgctagatcgtttccggtacgcgaaatggcgaaagctgttggcactagctgagaggcccttagttcgcagaggatgtgagagctgcggatacgggaacttcgcggacctgaatacggagcggaaatcaagtgcaggaattggatatccaaagcctcatggaaaagcggcgggactgctccttggggatcgaggtaactacggactggcgccctactcaaggtacaggagagatgcatcgagcctgcatagagagctacaaaacccacccatcgtcggacgtttgccgcgctccgcgagcttcaacgcagtcatcaccaaggccgacgaagcagttttctccgcgtagcagcttgagatcaacaaatacgtttggccctccacaccaggaatgttgactgtgttagatcagatcagtgagtcggaccggacgggtacgtcctggctgtcttcagcagctatccccttgccgtgggggtcgtggactcgtgcgttatcgagaacttgtgtcacctcacatcgacccttgctgagaggcgcgagcgttacccagccggttcatgacctaccaacaaacctgtcatccgtaccgcccatgccaaatgtggctccaggcgttcccgaaggaattactaagcgttccggcggtctacgagatctacagccaaatgggtgaaagaatttgcttgtttcctcctgttacctagcacagatcgcagggcggatggaccgcttcttcaccgtgcgataaaggaatcacctagggctcgcgctggtctcccgccggcgttctgtcgccaggcacgtaacaaggatgcgcagtctacggccatttcttgacgctcgttgctcatgaggggcgtcttcaaacgtgacgtgataagttctaaaggtccagcgttg

SEQ ID NO: 2 - DNA, 1000 bp, Artificial Sequence
  misc_feature
tatggtccgcaaggctgaacacggtggatatgcccgcaaaccccagtgaggccagcccggcacggatggtccgcaaacaattctgtagtttgtgaagtaggctagtcttccctggtaagacgatgcgttgttagtgagggcatacgtgcgaatcagcgttaacgagataaacgatgagcaatttgacgaatgctcttctcaactctcttggacatcgaggtgagtaagtgagcgacaaccgaacggcggtcagcatgtctagagtcacgcacattgactatccgttaatcacgtgtcgtttcggtaattccggccactgcactgaatgtctctgcaagcccgttgtttcgaagtcctctcatccattttgtgggagcgaaacagcgtttgcgcctgtcatatcactgcggcctagcgatccgggcgcctgttaggcatgtcaaacggctagttggtttgctcgttgcgactcaccgggtttcgttgtgaacaccgaggccggtgactagttcactgtcccgctatgtgttgacgatcatttccaagaggaacgcttagcagctcggtttccggcccggagtgactttttcttgacggttggcggacgacgcctcctggttcccacgcttacttgaatgtgttgtgagaaggcgatggcgaggacctgcctagtacagcggggactctctggaaacccgcatttactgaagactgttcccaggaacagcgatttctttctcacggtttaagagagaggctctttgtcagctcctgtgaatgggcaaagcatcgcgggacaactggaagacagacgtacccccactcgcttccctatgactctgaactcaaaagtttggctttggtaagtcgtgtttgtgtgtcttgggtctccaatgtccgaattgggtccgcacggggtctttcatagatacacgggggccgttggcccgactgcctctgtttgtacctcgatattaaaggggttccgttcaagtcatgt

SEQ ID NO: 3 - DNA, 534 bp, Synechococcus PCC7002
actgccgcactcaatgttcaaccagatctagtgattatttcggggttagcagccgatgcgggcaacctggtgaagcaactgcgagaattaggttacaacggcattattgttggggggaacggcttaaatacttctaatattttccccgtctgccaagcaaaatgtgatggggtgttggtggcccaagcctacagtgccgagttagataatgagattaaccgcgcgtttcgggacgcctattttcaacaaaaccaaaaagagccgccccaatttagtgcccaggcttttacggcgatccaagtttttgttgaagccctcagcagcctcgatgaaaaaacgcccttagaaactcttgctctaccggacttgcgacgacaactgcgggacgaaatttttgcaggtacctacgtcacgcctttgggtgaaatttccttcacagaggaaggggaaattgtccagaaggaattttttgtggcccaaattgaaatggatgaatcgggtcaacaggggcgtttcgccttcattgaaacgaac

SEQ ID NO: 4 - DNA, 457 bp, Synechococcus PCC7002
ctagggagattcttctaaagcgagcagggtttgccaagtggcgatcgcctcagggaaccataaccaactttgttcgaggcggcgggggttgagctgggaacgatcgagatctagggcgatcgcccggagtttttcggctttccctacaagaatttcctggtcaacgtcggtgggttgcctttgggccaaatcgtacatactaatcgccaggcccgcataaatctccgcctggagggctggggggaaatcctgggtcgttgccgccgctaaggcttcataccaacgatcattggcggcttcgtaatctgcattgaggtaatggacaaaacccagggccatcaaaatttcggggtcgttgggctgttgggctagggccatgtcccaattgtcttgcaccgtggcgatcgccgttgcttgggttggcgatggtgggacggcagccaaagttgtttgccag

SEQ ID NO: 5 - DNA, 487 bp, Artificial Sequence
  misc_feature
ggacctaacgccttcagacctagcagctgtaacgtgacacggtgccacctgacgtctaagaaaccattattatcatgacattaaccttttgttatcaataaaaaaggccgcgatttgcggccttattgttcgtcttagttagttagcccttagtgactcgaattcgcggccgcttctagagtactagtagcggccgctgcaggagtcactaagggttagttagttagccaattattgaagacgcttaacagcgtctttttttgtttctggtctcccgctcactcaaaggcggtaatctcgagtcccgtcaagtcagcgcgcaataaaaaagcccccggaaggtgatcttccgggggctttctcatgcgttccaagttatgtatggaccggccgacagatcgtcaagattactataagattccgcattgcggacgatttaggcgcactatcgcgctcaagttgatcgaatgtatgccccggatatgac

SEQ ID NO: 6 - DNA, 1223 bp, Artificial Sequence
  misc_feature
gtgaaggccagagttttgacagctagctcagtcctaggtataatgctagctactagagtagtggaggttactagatggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactttagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtaactcggtaccaaattccagaaaagagacgctgaaaagcgtcttttttcgttttggtcc

SEQ ID NO: 7 - DNA, 878 bp, Artificial Sequence
  misc_feature
ttaacaagatgtaattgacataagtcccatcaccgttgtataaatgtgtggaattgtgagcggataacaatttcacacatactagagtagtggaggttactagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaactcggtaccaaattccagaaaagacacccgaaagggtgttttttcgttttggtcc

SEQ ID NO: 8 - DNA, 893 bp, Synechococcus PCC7942
gatccggcagccggcggagcgctgctttcttggcaagcggtcgccagccccaacgccagggctgccagcccgaaacagcggggcaaggcagcttggaagggcgatcgcagcacgggcatggcaatgtctctctgaaggaatgcagaccttattcgtacagccagggttgaatcgtgggggtccaatcacttagctctgctgggctaaaccagagagcaatttcctgttgtgctgtttcgattgcatccgagccatggatgatgttgcggccaatattgacaccaaaatcaccacggatggtgcccggttctgccgtcagcggattggtagcgccgatcaacttgcgagcagccgccacaacgccttcgccttccaagacgatcgccacgatcggcccagaggtgatgaactcgacgaggccattgaagaaggggcgctcgcggtggacagcatagtgctgttcggccagctcgcgactgggcttcagctgctttaggcccaccagtttgaagcctttttgctcaaagcggccgatgatcgtaccgaccaaaccccgctgaacgccatcgggcttgatggcaataaatgtgcgttccacagacatctagatagtcctcaagacgaggcaagcattgagcttgccttcctatggttcgggatcactgggattcttgacaagcgatcgcggtcacatcgctatctcttaggacttcgcagcgggcgagtcggattgacccggtagggatttcgccagatcaatgcccgtggtttgtttcagcttctccagcaagctagcgatttgggtagcgctgccttccccttcgccaatcacagtgatcgactccacgtcgatatctggcacggtgcctgaaagcgtgacgagcagggactcg

SEQ ID NO: 9 - DNA, 886 bp, Synechococcus PCC7942
ttccgcgatcgcccgtttacaagctgcctcagctggggcgatcacatcggcttgaagttgctgctgcacctgtttgatccgctcctgctgcacagggagttctgcttggctacgagcgacttcggtagcaatgtccgcttcagcttcggccaccaccgcttcgcgccgcgtcaacgcatcctgaatccggcgctcggcctcggcttgggcgatcgctacatcgcgatcgatccgacgcagggccgtgatcttgtcattttcggccgtttggatcgcagaggcagcctgggcatcggcttcagcaattcgggcatctcgctgcagatcagcccgctgcttgcgtccactagccgagagataaccgacctcatcggaaatgttctggacttgcagcgtatcgaggactagacccagctgctcaaggtcatcctccgcctcttccagcagacttttggcaaaggcaattttgtcctcgttgatctgctccggcgtgaggctggctaaaacaccacgcaagttgccttcgagggtctccttggcaatttgctcgatttccttacggtttttgccaagcagccgctcgatcgcgttgtggatggtcggttcttccccagcaatcttgatattggcaacgccttcaacagtcaggggaatgccgcccttggagaaggcattggaaacgcgcaactcaatgatcatgttggtcagatccatgcggagcgctttttccagcagaggtacccgcaggctgctgccgcccttgaccaagcgatagccaactcggcggccatcactactgcggcgactactgccagcaaagatcaaaatttcactgggttggcagatgtagtagagattgcgcaggactaagctgccagccccggcg

SEQ ID NO: 10 - DNA, 798 bp, Synechococcus PCC7942
cgtctgcaagaagccggtgcccatctcatttttgacgatatgcgactgctgcccagtctgctccaatcgtcgccaaaagataactccacagcattgcccaatccctaacccctgctcgcgccgcaactacacactaaaccgttcctgcgcgatcgctcttactgttgatggctcgtgcttaaaaacaatgcaaccctaaccgtttcagctggtgattttcggacgatttggcttacagggataactgagagtcaacagcctctgtccgtcattgcacacccatccatgcactggggacttgactcatgctgaatcacatttcccttgtccattgggcgagaggggaggggaatcttctggactcttcactaagcggcgatcgcaggttcttctacccaagcagtggcgatcgcttgattgcagtcttcaatgctggcctctgcagccatcgccgccaccaaagcatcgtaggcgggacgttgttgctccagtaaagtcttcgcccgtaacaatccccagcgactgcgtaaatccgcttcggcaggattgcgatcgagttgccgccacagttgtttccactgggcgcgatcgtcagctcccccttccacgttgccgtagaccagttgctctgccgctgcaccggccatcaacacctgacaccactgttccagcgatcgctgactgagttgcccctgtgcggcttcggcttctagcgcagctgcttggaactgcacacccccgcgaccaggttgtccttggcgcagcgcttcccacgctgagagggtgtagcccgtcacg

SEQ ID NO: 11 - DNA, 733 bp, Synechococcus PCC7942
ggtaaccccagcgcggttgctaccaagtagtgacccgcttcgtgatgcaaaatccgctgacgatattcgggcgatcgctgctgaatgccatcgagcagtaacgtggcaccccgcccctgccaagtcaccgcatccagactgaacagcaccaagaggctaaaacccaatcccgccggtagcagcggagaactacccagcattggtcccaccaaagctaatgccgtcgtggtaaaaatcgcgatcgccgtcagactcaagcccagttcgctcatgcttcctcatctaggtcacagtcttcggcgatcgcatcgatctgatgctgcagcaagcgttttccataccggcgatcgcgccgtcgccctttcgctgccgtggcccgcttacgagctcgtttatcgaccacgatcgcatccaaatccgcgatcgcttcccagtccggcaattcagtctggggcgtccgtttcattaatcctgatcaggcacgaaattgctgtgcgtagtatcgcgcatagcggccagcctctgccaacagcgcatcgtgattgcctgcctcaacaatctggccgcgctccatcaccaagatgcggctggcattacgaaccgtagccagacggtgagcaatgataaagaccgtccgtccctgcatcacccgttctagggcctcttgcaccaaggtttcggactcggaatcaagcgccgaagtcgcctcatccagaattaaaatgcgtggatc

SEQ ID NO: 12 - DNA, 798 bp, Synechococcus PCC7002
ccgattagaccctaaatttccagaattttgcctatttcgtctgattaataaggtaattgggaataggcagcctgggaaatagtcggcaactccgcatatttgccgaggatcgattgcaccattgcatagagacaagccactaaagtaccgagaaaggcaacatttgaaagggtttcaacgagaagactgccgcccccaaagccaaaaaagcgaattaaaatcccgaacaacaccagggcaatgtcaatcaaaatcgcctggagggtattgaagcgaatgaagtggctaattttgctgttgcgtaccaccgcagcatagagaatgaagaaaataatcagccctgcaaagggaaatgaataaaggcggattaggggttgtaaaggcacatagattaccgccaggatcggaaagcgcattaaaaacggcagaccgaagggcaaagcgtagacgaggggtaataggtaaggtaaggccccaaataaacgatctgtgggttcagtggtagtggacataaaactttttctgcaatggactagctcctattctacagagaagtcttgaagaagttctgtggcgatcgccagggccaaacaatgggaaaaattaaacgaaattcaggaattttgcggcgatttcccctgcatcctgacaaaaggcagtagaattgtaaacatttgttaatctctttttgaaactgaatgcaaagtacagtccgctccccagggagtcgcgagtctctacggcaagatctgcccttcaccctcaaggatgtgaaagcagccatcccagactactgtt

SEQ ID NO: 13 - DNA, 809 bp, Synechococcus PCC7002
gagccatgctcccatttggcggagtttctttagatcccaaaaggcttgtcactacatcgccgatcagggttctcacttgtattaccagccgaaaaaataagctccaaaagcgtgactagatagcgctgtaaaccctaattttcctcggagaattggggtttttctttggttggtgatgatgatttttagagagatcgcctatgatcgagaccgttgaatttatggcagcaacatgaaacgggttcgggtcaagtacagttcaccagtcttttggctgggtcttggtttaaccggcattttgggcattggggcgatcgccaaccgcaatgctttacttcccttgtttccaggcagtggtcaggtggaatctccagcagcggtagattctgaagtgttagccctggtggatctcgccccggcagagcggcgcgatcgcctcgaagtgatcgccaacagcagcaacaatgacctcgaccgaaaccgcgcccgttacctgttggggatggactacttggttgcggaagatggggcagcggccctagccgccttcgagaacctagaacaggactatccggtgttaacgcctcatattttgattaaacggggccgcgcctacgaattggtcaataatcccgaacaggcccaggtgatttggtttgatgtggtgcaaaattatccagaggatgctgccgctgccgaagcgctgtttcgcctcagtgcctacgacccgaaatatgccgaccaggcgatcgccgaatatcccgcccatccgcgcacccaaagcttgatccaacaacgccttgcagaaa

SEQ ID NO: 14 - DNA, 623 bp, Synechocystis PCC6803
ggcaatgcccactcctccacaggcggtgagggtaaagctcagcagtaaagttaccaggactacagcagtggatagggactttttcaacatgggagaaagggaagaactgggcaagaaggcaaaattacctttccttacccattaaacctccaatggttgaccagaaactagaggtagaatgattcccgccagaaaaagaagtttaacaatttgccatgggcactgttctgaagcggggaggaaggctatggcctcctcaaccaatttatttcctgcctaacccaggggacgttgttgaggctataagttgaggctataaatttaacttattaaaaggttcgacaaatttgagatagttttgtggcaaagatactgcttaggaaccaaatattgcataaacttagagatatagttttttctaaaaaaatagtcttatttctatctattgaatcggggcaatttaaactcagaatagattagttgttcccagctgaaaccatcgtgtgctttttccagaggcgtttttggcaatttttcctctggtaaatttcaccgactttggggcaatgctcataatcaccatagagtgaaatccatgaacaagtttgaatcaagacaatcgg

SEQ ID NO: 15 - DNA, 587 bp, Synechocystis PCC6803
gctctttttcctgggttgtggtgtcggcttactactgtcggtggtttgggtcaatgttgctcgccatagtcctccgctagaatcctccccagtcaaggtctcgcctcccttccaggtcgactagtcacaacaatttaaaaatcagaaaaattgtcccattgatcaacttacagggggccattgagcaaaatccggggtcaccatctagtccccaaaaagctggcgatcgccaaataatagtaaaacttatcattcaaatttaaaattacttagcagatccagggggacaactgcaaaattggtcggatttacatatagactttagcttatagatttcaagacataggcattcaaacctgcatagacaagagtctatacagagcgaagccaatggggttcattgcccctggaaagatcaagcaaactgccgaagattcagggccaagctttactaccccaatccccataaatttcaaccaaggagacaatttacattatggattttttgtccaatttcttgacggacttcgtgggacaattgcagtccccaaccctagcctttctgattggggggatggttattgccg

SEQ ID NO: 16 - DNA, 501 bp, Synechocystis PCC6803
atgactattcaatacacccccctagccgatcgcctgttggcctacctcgccgccgatcgcctaaatctcagcgccaagagtagttccctcaacaccagtattctgctcagcagtgacctattcaatcaggaagggggaattgtaacagccaactatggctttgatggttatatgggaattcccggtatggatggcaccgatgcggaatcccaacagattgcctttgacaacaatgtggcctggaataacctgggggatttgtccaccaccacccaacgggcctacacttcggctattagcacagacacagtgcagagtgtttatggcgttaatctggaaaaaaacgataacattcccattgtttttgcgtggcccatttttcccaccacccttaatcccacagattttcaggtaatgcttaacacgggggaaattgtcaccccggtgatcgcctctttgattcccaacagtgaatacaacgaacggcaaacggtagtaatt

SEQ ID NO: 17 - DNA, 522 bp, Synechocystis PCC6803
acccagatgggaaggtattttatgcttcctttgctgccgctgatgaccaagccacggatttaaccacggcgatcgccaatcccacggccatcgatttaattaacgccaggggatttacggcgggtagttccgtcaccgtatcgggttcctacagtcgggaagccttttttgatggatccatgggtttttatcgacttctggacgataacggtgcagtgctagatcccttaacaggtggtgtaatcaacccaggacaggtaggttatcaagaagcagctttggcagatagcaatcgtttgcaagccactggctccaccctaacggcagaagacctagaaaccagagcattttccttcaatattttgggtggcgagttgtatgcgccatttttaacggttaatgacagtctttccggtattaatcagacttattttgcctttgggtcggccaacccagatggcatcagccacagcacaaacttgggacccaacgtgattggttttgaagattttctcggcggag

SEQ ID NO: 18 - DNA, 20 bp, Artificial Sequence
  primer_bind
acagccgtagactacaacgg

SEQ ID NO: 19 - DNA, 20 bp, Artificial Sequence
  primer_bind
gtgtcacgttacagctgcta

(19) United States
(12) Patent Application Publication
(10) Pub. No.: US 2023/0365982 A1
(43) Pub. Date: Nov. 16, 2023

(54) STANDARDISED CYANOBACTERIAL STRAIN ENGINEERING

(71) Applicant: BONDI BIO PTY LTD, Sydney, New South Wales (AU)

(72) Inventors: James BROWN, Sydney, New South Wales (AU); Cameron O'BRIEN, Sydney, New South Wales (AU); Artur WLODARCZYK, Sydney, New South Wales (AU); Dominik KOPP, Sydney, New South Wales (AU)

(21) Appl. No.: 18/246,564

(22) PCT Filed: Sep. 24, 2021
(86) PCT No.: PCT/AU2021/051121

§ 371 Date: Mar. 24, 2023

(30) Foreign Application Priority Data
Sep. 25, 2020 (AU) 2020903460

(51) Int. Cl. C12N 15/74; C12N 15/11; C12N 15/65

(52) U.S. Cl. CPC C12N 15/74; C12N 15/11; C12N 15/65

(57) ABSTRACT

There is provided nucleic acid constructs comprising two portions of a neutral site and an intervening heterologous nucleic acid comprising landing zones and a selectable marker for producing standardised cyanobacteria cell strains for stain engineering. There is also provided methods for standardised strain engineering using nucleic acid cassettes comprising combinations of landing zones.

### TECHNICAL FIELD

The technology relates to nucleic acids and methods for producing recombinant cells such as cyanobacteria suitable for standardised strain-engineering. The technology also relates to methods of strain engineering.

### CROSS REFERENCE TO RELATED APPLICATION

This application claims the benefit of Australian Provisional Application No. 2020903460 filed 25 Sep. 2020, the entire content of which is incorporated by reference herein.

### BACKGROUND

Many cyanobacteria are naturally-competent or can be transformed using well known methods. For many years, researchers have routinely exploited this natural cellular DNA-uptake phenomenon or artificially induced competence to chromosomally-integrate new genes via homologous recombination.

Despite many years of genetic modification, DNA insertions in cyanobacteria have typically been limited to ad hoc single or double-insertions into a small number of strain-specific ‘Neutral Sites’ which are intergenic locations within a strain's chromosome that show no observable phenotypic consequence of foreign DNA insertion.

Using constructs containing flanking homologous DNA regions of the Neutral Sites, typically of 400-800 bp, researchers are routinely able to insert genetic cassettes and modify the strain's chromosomal DNA. However, most cyanobacteria research is conducted in a single species, there is little incentive or benefit to move beyond the small set of naturally-occurring ‘Neutral Sites’ and attempt to standardise or engineer a broadly-applicable solution. In addition, Neutral Sites are species or strain-specific and little or no work has been completed to identify regions that are cross-species compatible.

Cyanobacteria produce a large number of secondary metabolites and have the potential to be used in the production of pharmaceuticals, high value chemicals and as tools for bioremediation. The diverse array of biochemical pathways of cyanobacteria are apparent in the more than 400 cyanobacterial genomes available in public databases (Alvarenga et al, Front. Microbiol., volume 8, 2017, page 809). Consequently, the potential to improve and/or modify metabolite production by employing genetically manipulated cyanobacteria is being explored but is limited by the need to re-engineer constructs to be specific for each strain.

Accordingly, there is a need to develop new interspecies ‘landing-zones’, and cells containing the interspecies ‘landing-zones’ for standardised strain engineering of cyanobacteria.

### SUMMARY

In a first aspect, there is provided a nucleic acid cassette comprising;
two portions of a neutral site sequence; and
a heterologous nucleic acid comprising at least two landing zones and a first selectable marker gene located between the landing zones; and
wherein the heterologous nucleic acid is between the two portions of the neutral site.

The neutral site may be substantially homologous to at least a part of a non-essential region of a microorganism genome.

The non-essential region may be selected from the NSC1 region of Synechocystis sp. strain PCC 6803, the slr0168 region of Synechocystis sp. strain PCC 6803, the A0159 region of Synechocystis sp. strain PCC 7002, the A2842 region of Synechocystis sp. strain PCC 7002, or a non-essential region of Synechocystis sp. strain PCC 7942.

The length of each neutral site sequence portion may be independently selected from about 500 bp, about 550 bp, about 600 bp, about 650 bp, about 700 bp, about 750 bp, about 800 bp, about 850 bp, about 900 bp, about 950 bp, or at least about 1000 bp.

In one embodiment the landing zone comprises a core sequence consisting of a randomly generated nucleic acid sequence with a GC content of approximately 50% and lacking a bacterial promoter sequence.

The landing zone may further comprise at least one transcriptional terminator and at least one translational insulator, preferably the landing zone comprises a transcriptional terminator and a translational insulator at either end of the core sequence.

In some embodiments the landing zone comprises or consists of SEQ ID NO: 1 or SEQ ID NO: 2.

In some embodiments the neutral site may be selected from any one of SEQ ID NOs: 8-17, that is the neutral site portions are selected from within each sequence

The neutral site portions may be SEQ ID NO: 3 and SEQ ID NO: 4.

In cassettes having multiple landing zones, each landing zone may be the same of different same or different.

The selectable marker gene may be selected from genes that confer resistance to bleomycin, chloramphenicol, erythromycin, kanamycin, spectinomycin, neomycin, streptomycin, zeocin or gentamicin.

In a second aspect there is provided a cell comprising the nucleic acid cassette of the first aspect, preferably the nucleic acid cassette is integrated into the genome of the cell.

In a third aspect the heterologous nucleic acid comprises, in a 5′ to 3′ direction a first landing zone, a second landing zone, a first selectable marker, a third landing zone, and a fourth landing zone.

In a fourth aspect there is provided a cell comprising the nucleic acid cassette of the third aspect, preferably the nucleic acid cassette is integrated into the genome of the cell.

In a fifth aspect there is provided a method for generating a recombinant cell comprising a nucleic acid of interest, the method comprising:
contacting the cell of the second aspect with a nucleic acid insert under conditions that allow recombination of the nucleic acid insert with the nucleic acid cassette in the genome of the cell; wherein
the nucleic acid insert comprises the nucleic acid of interest and a second selectable marker flanked by two landing zones, each landing zone comprising a sequence at least 90% identical to the landing zones in the cell's genome.

The method may further comprise culturing the cell in the presence of a selection agent for the second selectable marker, thereby selecting a recombinant cell comprising the second selectable marker and the nucleic acid of interest.

In a sixth aspect there is provided a method for generating a recombinant cell comprising a first nucleic acid of interest, the method comprising:
(a) contacting the cell of the fourth aspect with a first nucleic acid insert under conditions that allow recombination of the a first nucleic acid insert with the nucleic acid cassette in the genome of the cell; wherein the first nucleic acid insert comprises, in a 5′ to 3′ direction, the first landing zone, the first nucleic acid of interest, a fifth landing zone, a second selectable marker, and the third landing zone, or wherein the first and/or third landing zones have at least 90% sequence identity to the first and/or third landing zones in the cell, respectively; and
(b) culturing the cell in the presence of a selection agent for the second selectable marker, thereby selecting a recombinant cell comprising the second selectable marker and the first nucleic acid of interest.

The method may further comprise (c) contacting a cell from step (b) with a second nucleic acid insert under conditions that allow recombination of a second nucleic acid insert with the nucleic acid construct in the genome of the cell; wherein
the second nucleic acid insert comprises, in a 5′ to 3′ direction, the fifth landing zone, a further selectable marker, a sixth landing zone, a second nucleic acid of interest, and the fourth landing zone, or wherein the fourth and/or fifth landing zones have at least 90% sequence identity to the fourth and/or fifth landing zones in the cell, respectively; and
(d) culturing the cell in the presence of a selection agent for the further selectable marker, thereby selecting a recombinant cell comprising the further selectable marker, the first nucleic acid of interest and the second nucleic acid of interest.

In a seventh aspect there is provided a method for generating a recombinant cell comprising a first nucleic acid of interest, the method comprising:
(a) contacting the cell of the fourth aspect with the first nucleic acid insert under conditions that allow recombination of the a first nucleic acid insert with the nucleic acid construct in the genome of the cell; wherein the first nucleic acid insert comprises, in a 5′ to 3′ direction, the second landing zone, a second selectable marker, a sixth landing zone, the first nucleic acid of interest, and the fourth landing zone, or wherein the second and/or fourth landing zones have at least 90% sequence identity to the second and/or fourth landing zones in the cell, respectively;
(b) culturing the cell in the presence of a selection agent for the second selectable marker, thereby selecting a recombinant cell comprising the second selectable marker and the first nucleic acid of interest.

The method of claim may further comprise (c) contacting a cell from step (b) with a second nucleic acid insert under conditions that allow recombination of the second nucleic acid insert with the nucleic acid construct in the genome of the cell; wherein the second nucleic acid insert comprises, in a 5′ to 3′ direction, the first landing zone, a second nucleic acid of interest, the fifth landing zone, a further selectable marker, and the sixth landing zone, or wherein the first and/or sixth landing zones have at least 90% sequence identity to the first and/or sixth landing zones in the cell, respectively; and
(d) culturing the cell in the presence of a selection agent for the further selectable marker, thereby selecting a recombinant cell comprising the further selectable marker, the first nucleic acid of interest and the second nucleic acid of interest.

The nucleic acid of interest, first nucleic acid of interest, or second nucleic acid of interest may be operatively coupled to a constitutive or inducible promoter.

The cell may be a Cyanobacteria, for example a Cyanobacteria is selected from the group consisting of Synechocystis sp. PCC 6803, Synechococcus elongatus PCC 7942, Synechococcus sp. PCC 7002, Synechococcus sp.PCC 11901, Synechococcus sp. UTEX 2434, Synechococcus sp. UTEX 2973, Synechococcus sp. UTEX 3153, Synechococcus sp. UTEX 3154, Anabaena variabilis PCC 7120, and Leptolyngbya sp. BL0902.

### DEFINITIONS

Throughout this specification, unless the context clearly requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Throughout this specification, the term ‘consisting of’ means consisting only of.

Any discussion of documents, acts, materials, devices, articles or the like which has been included in the present specification is solely for the purpose of providing a context for the present technology. It is not to be taken as an admission that any or all of these matters form part of the prior art base or were common general knowledge in the field relevant to the present technology as it existed before the priority date of each claim of this specification.

Unless the context requires otherwise or specifically stated to the contrary, integers, steps, or elements of the technology recited herein as singular integers, steps or elements clearly encompass both singular and plural forms of the recited integers, steps or elements.

In the context of the present specification the terms ‘a’ and ‘an’ are used to refer to one or more than one (i.e., at least one) of the grammatical object of the article. By way of example, reference to ‘an element’ means one element, or more than one element.

In the context of the present specification the term ‘about’ means that reference to a figure or value is not to be taken as an absolute figure or value, but includes margins of variation above or below the figure or value in line with what a skilled person would understand according to the art, including within typical margins of error or instrument limitation. In other words, use of the term ‘about’ is understood to refer to a range or approximation that a person or skilled in the art would consider to be equivalent to a recited value in the context of achieving the same function or result.

Those skilled in the art will appreciate that the technology described herein is susceptible to variations and modifications other than those specifically described. It is to be understood that the technology includes all such variations and modifications. For the avoidance of doubt, the technology also includes all of the steps, features, and compounds referred to or indicated in this specification, individually or collectively, and any and all combinations of any two or more of said steps, features and compounds.

In order that the present technology may be more clearly understood, preferred embodiments will be described with reference to the following drawings and examples.

### BRIEF DESCRIPTION OF THE FIGURES

FIG. 1: illustrates the prior art technique for DNA insertion to neutral sites. Using flanking homologous DNA regions of 400-800 bp, heterologous DNA can be inserted into the genome of the naturally-competent cyanobacterial strain's chromosomal DNA. This is achieved through the insertion of a Genetic Cassette (GC) comprising a nucleic acid of interest and a Selection Marker (SM) into Neutral Sites (NS) which are intergenic locations within a strain's chromosome that show no observable phenotypic consequence of heterologous DNA insertion.

FIG. 2: illustrates one method to create universal ‘Landing Zones’ within WT strains. Wild-type strains of naturally-competent cyanobacteria are initially engineered to include universal Landing Zones (LZ). A heterologous DNA section containing a Selection Marker (SM), flanked by the prefix and suffix LZ, in-turn flanked by the Neutral Site (NS) DNA regions, are chromosomally-integrated via homologous recombination to produce a ‘Chassis Strain’. A new interspecies Genetic Cassette (GC) is then constructed, with an alternative SM and flanked by prefix and suffix LZ, and can be integrated into the Chassis strain in a second-round of DNA integration.

FIG. 3: Cross-species compatibility via the of engineering of universal Landing Zones. DNA insertions have typically been limited to ad hoc insertions into a small number of strain-specific Neutral Sites (NS), with no attempt to identify regions that are cross-species compatible. When engineering multiple strains (A, B, C) with the same Genetic Cassette (GC), new sections of DNA must first be constructed that includes strain-specific NS homologous regions flanking the GC (a, b, c). Using the universal Landing Zone, a single section of heterologous DNA can be integrated into multiple species via the engineered Chassis Strains (A*, B*, C*).

FIG. 4: DNA ‘Resistance-Pivot’ insertion into Neutral Site. Wild-type strains of naturally-competent cyanobacteria are initially engineered to include x4 universal Landing Zones (LZ #). A heterologous DNA section containing a Selection Marker (SM), flanked either side by x2 Landing Zones (Left: LZA, LZB1; Right: LZC1, LZD), and in-turn, flanked by the Neutral Site (NS) DNA regions, are chromosomally-integrated via homologous recombination to produce a ‘Resistance-Pivot Chassis Strain’.

FIG. 5: Creation of universal ‘Landing Zones’ within WT strains. Wild-type strains of naturally-competent cyanobacteria are initially engineered to include x4 universal Landing Zones (LZA, LZB1, LZC1, LZD) and Selection Marker (SM1), as described in FIG. 4. (i) and (ii): A new interspecies Genetic Cassette (GCA, e.g. SEQ ID NO: 7) can be integrated either side of SM1 by using an alternative selection marker (SM2). To prevent only the SM being replaced, it is necessary to exchange LZs and replace LZB1 and LZC1 with new alternate internal Landing Zones ((i)LZB2; (ii)LZC2). GCA can be integrated into the Chassis Strain between LZA in a second-round of DNA integration.

FIG. 6: Universal ‘Landing Zones’ and marker-enabled flip (i) Selection Marker SM2 was successfully integrated into an engineered strain and exchanged for Selection Marker SM1, using homologous recombination via the universal Landing Zones LZA (e.g. SEQ ID NO: 1) and LZB (e.g. SEQ ID NO: 2). (ii) Genetic Cassette GCA (e.g. SEQ ID NO: 7) and Selection Marker SM2 (are to be) integrated into an engineered strain and exchanged for Selection Marker SM1, using homologous recombination via the universal Landing Zones LZA (e.g. SEQ ID NO: 1) and LZB (e.g. SEQ ID NO: 2). NS =Neutral sites (e.g. SEQ ID NO: 3 and SED ID NO: 4).

FIG. 7: Agarose gel showing segregated strains after marker-enabled ‘flip’ Colony PCR was performed on newly-transformed colonies of strain SHL5-232. SM2 (within SHL5-232) is ˜500 bp shorter than SM1(within SHL5-231), and hence an identifiable length shift can be observed when compared to parent strain SHL5-231.

FIG. 8: Plasmid maps of (A) pBB-SHL5-231 including GCB (SEQ ID NO: 5) and (B) pBB-SHL5-232.

FIG. 9: Agarose gel showing segregated strains after marker-enabled ‘flip’ Colony PCR was performed on newly-transformed colonies of strain TMR1-237. Insertion of SM2 and GCA (integrated via pBB-TMR1-237) is ˜600 bp longer than SM1 alone (within TMR1-231), and hence an identifiable length shift can be observed when compared to parent strain TMR1-231.

FIG. 10: Plasmid maps of (A) pBB-TNR-1-231 including genetic cassette B (GCB, SEQ ID NO: 6) and (B) pBB-TMR1-237 including genetic cassette A (GCA, SEQ ID NO 7).

FIG. 11: Each Landing Zone (LZ) contains Transcriptional Terminators (TER) and translational insulators (TLT) at both the 3′ and 5′ ends to avoid any potential readthrough to/from inserted Genetic Cassettes in engineered strains

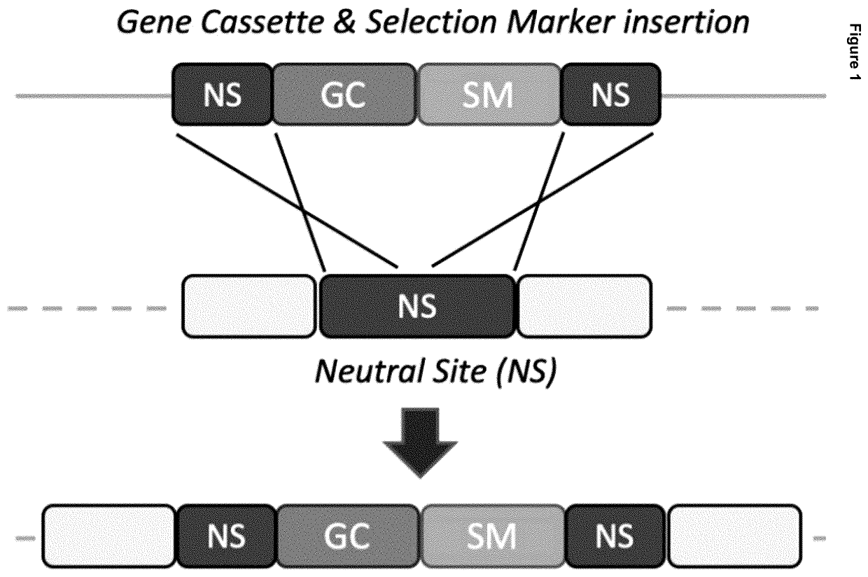

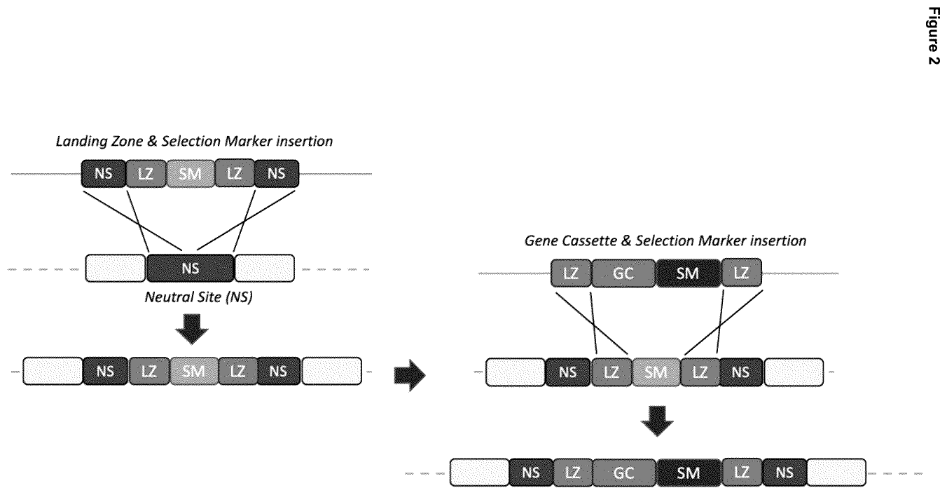

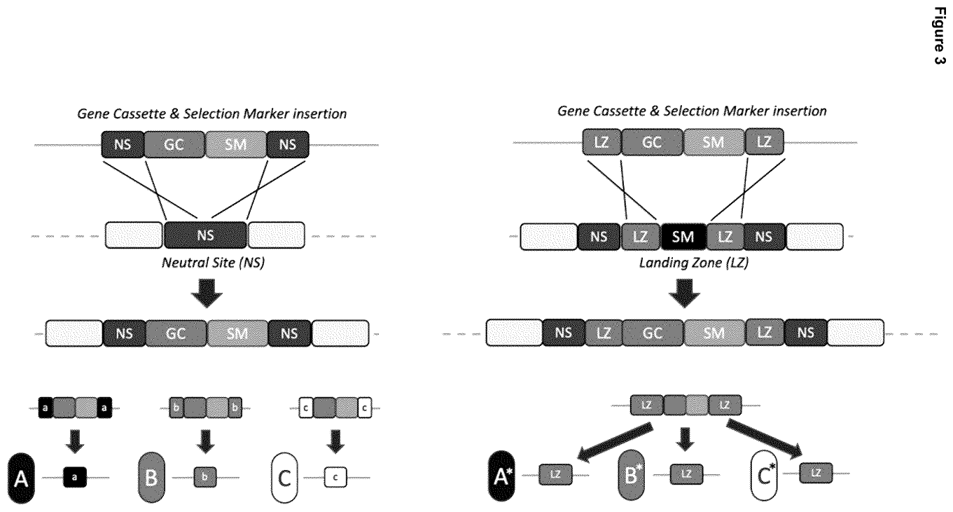

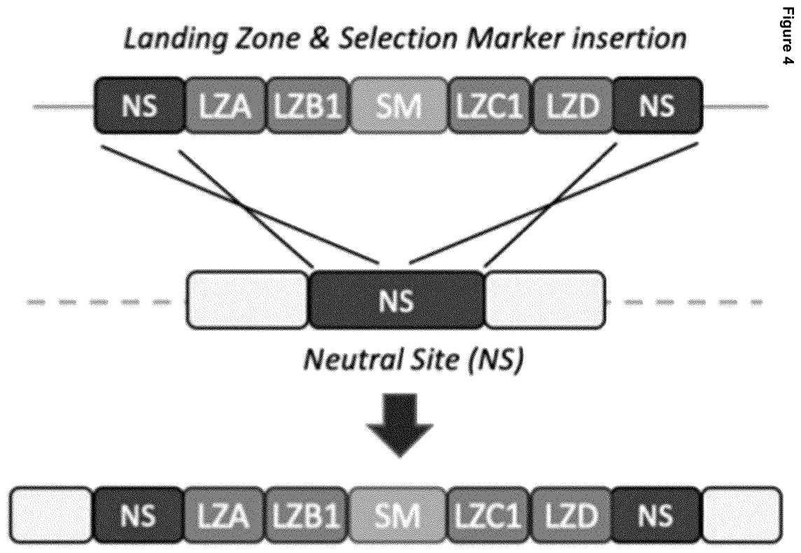

### DESCRIPTION OF EMBODIMENTS

The present inventors have developed nucleic acids and methods of using the nucleic acids to facilitate the rapid and simple engineering of multiple species of microorganisms, particularly cyanobacteria, using standardised interspecies DNA cassettes in combination with universal ‘Landing Zones’. By first manipulating wild-type strains and integrating standardised sections of DNA within existing Neutral Sites to create a ‘Landing Zone’, interspecies-compatible DNA ‘Insertion Cassettes’ can be constructed and introduced across multiple strains, without the need to rebuild or transfer the insertion to an appropriate strain-specific integration vector.

Wild-type strains of naturally-competent cyanobacteria are initially engineered to include a set of universal ‘Landing Zones’ as follows:
a. Existing or new ‘Neutral Sites’ within wildtype strains are identified
b. DNA cassettes containing the universal ‘Landing Zones’ (LZs), flanked by the matching ‘Neutral Site’ (NS) DNA regions, are chromosomally-integrated via Homologous Recombination using antibiotic selection to produce a (set of) ‘Chassis Strain’(s).
c. New interspecies DNA ‘Insertion Cassettes’ (ICs) are constructed, with core DNA insertions flanked by prefix and suffix LZs. Due to the standardisation of LZs, these ICs are now strain-agnostic and compatible with any ‘Chassis Strain’.

Strain development often takes an iterative approach, whereby further modifications are incorporated by the insertion of additional genetic cassettes. Previously this required use of a separate neutral site (NS) for each modification. Given that there are typically only 2 or 3 known neutral sites in each strain this limits the number of modifications that can be made.

The inventors have developed the use of universal Landing Zones (LZs) to design a nucleic acid construct comprising a nucleic acid of interest, strain-independent Landing Zones, and a Selection Marker (SM).

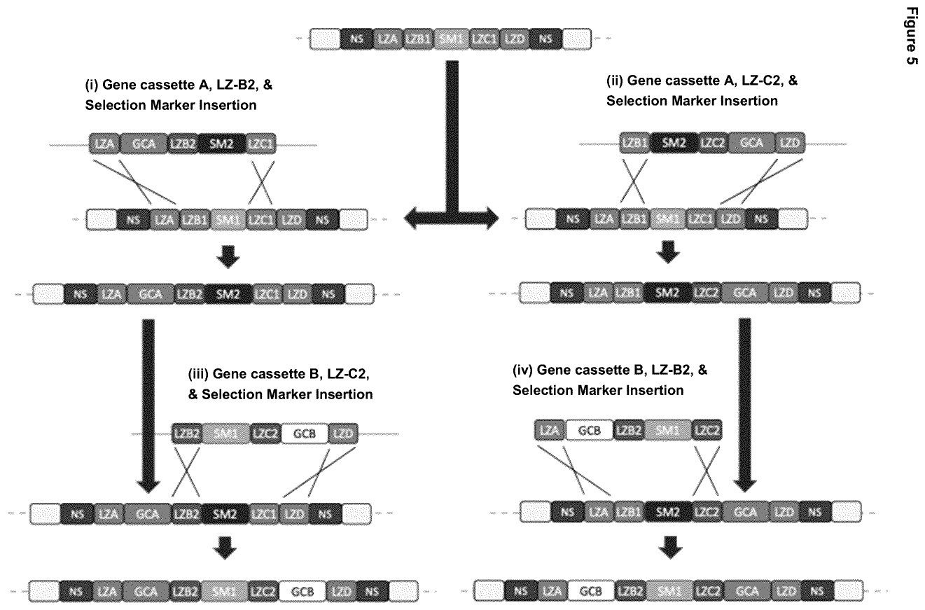

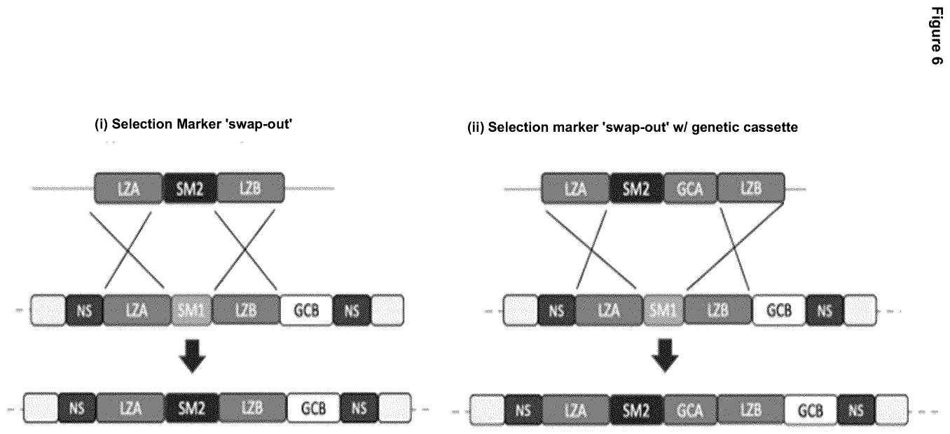

In one embodiment four LZs are used (see FIG. 4). The central SM can then be exchanged for a second SM to integrate a second nucleic acid of interest into the engineered strain (see FIG. 5). This marker-enabled exchange or “flip” method can then be used repeatedly to integrate cassettes ad infinitum. That is, the “flip” can continue indefinitely, with each flip changing the selectable marker. However, after the first two flips, previously integrated cassettes are removed in the exchange, assuming additional landing zones are not inserted.

Critically, with reference to FIG. 4, the two internal LZs, directly either side of the selection marker (LZB1/LZC1), must be exchanged for alternative an LZ (LZB2/LZC2) each time a cassette is ‘flipped-in’, as demonstrated and described in detail in FIG. 5.

In one aspect the invention provides a nucleic acid construct comprising two portions of a neutral site. A heterologous nucleic acid is inserted between the neutral site portions. The heterologous nucleic acid can comprise two landing zones separated by a selectable marker. This nucleic acid construct can be transformed into a microorganism and combine with the genome of the microorganism, which contains a neutral site having a corresponding sequence to the portions of the neutral site of the nucleic acid construct. This occurs via homologous recombination.

Accordingly, there is provided a microorganism or ‘chassis strain’ comprising the nucleic acid construct integrated into the genome of the microorganism such that the genome of the ‘chassis-strain’ comprises the neutral site portions, landing zones and selectable marker of the nucleic acid construct.

The term ‘homologous recombination’ refers to the introduction of a nucleic acid fragment of interest into a genome by the process of strand exchange that can occur spontaneously with the alignment of homologous nucleic acid sequences (i.e. sets of complementary strands). As is known in the art, microorganisms are efficient at homologous recombination. Methods and conditions allowing homologous recombination are well known in the art. Thus, in general, the neutral site portions (and landing zones) in the nucleic acid constructs disclosed herein function in pairs (for example the neutral site portions). The first member of the pair is located 5′ to an intervening nucleic acid sequence typically comprising a nucleic acid of interest and a selectable marker. The second member of the pair is located 3′ to the intervening nucleic acid sequence.

The neutral sites can be designed to be homologous to any region of the cyanobacterial genome and a skilled person can design the neutral sites using methods known in the art.

In some embodiments the length of the neutral site portions are at least 500 bp. For example, the length of each neutral site portion may be independently selected from about 500 bp, 550 bp, 600 bp, 650 bp, 700 bp, 750 bp, 800 bp, 850 bp, 900 bp, 950 bp, or at least about 1000 bp.

In some embodiments the neutral site may be selected from any one of SEQ ID NOs: 8-17, that is the neutral site portions are selected from within each sequence.

In one embodiment the neutral site portions may be SEQ ID NO: 3 and SEQ ID NO: 4. SEQ ID NO: 3 is a portion of a PCC 7002 non-essential region, found in GenBank accession ACA99827). SEQ ID NO: 4 is also a portion of a PCC 7002 non-essential region, found in GenBank accession ACA99827

The neutral sites can be homologous to any region of the microbial genome is not required for viability and can therefore be tailored using methods known in the art. In some embodiments the neutral sites are homologous to non-essential regions. Non-essential regions are known in the art.

Suitable non-essential regions include the following:

i.
SEQ ID NO: 8
(HRR-10001 (PCC 7942 Neutral Site, GenBank AAA81020))
ii.
SEQ ID NO: 9
(HRR-10002 (PCC 7942 Neutral Site, GenBank AAA81020))
iii.
SEQ ID NO: 10
(HRR-10003 (PCC 7942 Neutral Site, GenBank AAA86649))
iv.
SEQ ID NO: 11
(HRR-10004 (PCC 7942 Neutral Site, GenBank AAA86649))
v.
SEQ ID NO: 12
(BB-HRR-20001 (PCC 7002 Neutral Site, A0159 Knock-Out))
vi.
SEQ ID NO: 13
(BB-HRR-20002 (PCC 7002 Neutral Site, A0159 Knock-Out))
vii.
SEQ ID NO: 14
(BB-HRR-30001 (PCC 6803 Neutral Site NSC1/GenBank QWO81945))
viii.
SEQ ID NO: 15
(BB-HRR-30002 (PCC 6803 Neutral Site NSC1/GenBank QWO81945))
ix.
SEQ ID NO: 16
(BB-HRR-30003 (PCC 6803 Neutral Site slr0168/GenBank QWO79510))
x.
SEQ ID NO: 17
(BB-HRR-30004 PCC 6803 Neutral Site slr0168/GenBank QWO79510))

Suitable non-essential regions for PCC 6803 are described as the NSC1 site by Ng, A. H., Berla, B. M. and Pakrasi, H. B., 2015. Fine-tuning of photoautotrophic protein production by combining promoters and neutral sites in the Cyanobacterium Synechocystis sp. strain PCC 6803. Appl. Environ. Microbiol., 81(19), pp. 6857-6863.

In another embodiment the non-essential site may for PCC 6803 may be slr0168 as described by the Xiao, Y., Wang, S., Rommelfanger, S., Balassy, A., Barba-Ostria, C., Gu, P., Galazka, J. M. and Zhang, F., 2018. Developing a Cas9-based tool to engineer native plasmids in Synechocystis sp. PCC 6803. Biotechnology and bioengineering, 115(9), pp. 2305-2314.

For PCC 7002 non-essential sites such as A0159 and A2842 may be used, these sites are described in Vogel, A. I. M., Lale, R. and Hohmann-Marriott, M. F., 2017. Streamlining recombination-mediated genetic engineering by validating three neutral integration sites in Synechococcus sp. PCC 7002. Journal of biological engineering, 11(1), p. 19.

Non-essential sites suitable for PCC 7942 are described in Kulkarni, R. D. and Golden, S. S., 1997. mRNA stability is regulated by a coding-region element and the unique 5′ untranslated leader sequences of the three Synechococcus psbA transcripts. Molecular microbiology, 24(6), pp. 1131-1142; and Andersson, C. R., 2000. Application of bioluminescence to the study of circadian rhythms in cyanobacteria. Methods Enzymol., 305, pp. 527-542

### Landing Zones

The landing zones are artificial sequences designed to be amenable to homologous recombination. Landing zones comprise a core sequence (LZ core) that mirrors the GC content of native cyanobacterial DNA which has a GC content of about 50%. Accordingly, the GC content of the LZ core is from about 40% to about 60%. Ideally the landing zone sequences are not transcribed and therefore contain at least one transcriptional terminator sequence (TER), at least one translational insulator sequence (TLT), or both.

In a preferred embodiment the landing zone comprises a TER and a TLT at either side of the LZ core.

LZ cores can be produced by randomly generating DNA sequences of approximately 50% GC content. Any sequences containing bacterial promoters can be altered or discarded. The LZ core may be about 50 bp to about 150 bp, for example the LZ core may be 50 bp, 60 bp, 70 bp, 80 bp, 90 bp, 100 bp, 110 bp, 120 bp, 130 bp, 140 bp, or 150 bp. In some embodiments the LZ core is about 100 bp.

Any known transcriptional terminators (TER) sequences can be used in the landing zones.

Suitable translational insulators (TLT), include stop-codons in all six reading frames, were created and screened, removing repetitive parts.

The TLT and/or TER sequences can be added to the 5′ and/or 3′ ends of the LZ core to generate a landing zone.

Each landing zone is a nucleic acid sequence which is unique to the nucleic acid construct in that the nucleic acid construct does not comprise another landing zone of the same sequence, and the landing zone sequence is not found in the wild type organism used in the methods.

Preferably, the landing zones are each about 150 bp, about 200 bp, about 250 bp, about 300 bp, about 350 bp, about 400 bp, about 450 bp, about 500 bp, about 550 bp, about 600 bp, about 650 bp, about 700 bp, about 750 bp, about 800 bp, about 850 bp, about 900 bp, about 950 bp, or at least about 1000 bp.

In some embodiment the landing zone may consist, consist essentially of or comprise SEQ ID NO; 1 or SEQ ID NO: 2.

### Methods

There are also provided methods for generating a recombinant microorganism containing a nucleic acid of interest from the chassis-strain. In general the methods comprise transforming the chassis-strain with a nucleic acid insert comprising the nucleic acid of interest under conditions that allow recombination of the gene cassette with the nucleic acid construct in the genome of the microorganism.

The nucleic acid insert comprises a nucleic acid of interest (either alone or operably linked to a promoter) and a selectable marker that is different to the selectable marker in the chassis strain, these elements are flanked by two landing zones, each landing zone comprising a sequence at least 90% identical to the landing zones in the chassis strain.

It is noted that absolute homology between the landing zones in the chassis strain and the corresponding landing zones in the gene cassette is not required as homologous recombination can occur between sequences that do not exactly match. For example the landing zones in the nucleic acid insert may be about 90%, 91%, 92%, 93%, 94%, 95%, 96%, 97%, 98%, 99%, or 100% identical to the landing zones in the chassis-strain.

Previously, the insertion of the same nucleic acid of interest into multiple strains would require the preparation of different constructs for each strain, each having the nucleic acid of interest and neutral-site portions unique to each strain. The present invention provides chassis strains containing universal landing zones that allow, in this example, the preparation of a single nucleic acid insert that can be transformed into multiple chassis-strains.

In some aspects the nucleic acid construct comprises two portions of a neutral site with an intervening heterologous nucleic acid comprising, in a 5′ to 3′ direction, a first (eg LZA in FIG. 4), second (e.g. LZB1 in FIG. 4), third (e.g. LZC1 in FIG. 4), and fourth (e.g. LZD in FIG. 4) landing zone, and a selectable marker between the second and third landing zones. In this aspect the heterologous nucleic acid is between the two portions of the neutral site.

Similar to the above this nucleic acid construct can be integrated into the genome of a microorganism to generate a ‘resistance-pivot chassis-strain’ wherein the genome of the chassis-strain comprises the neutral site portions, multiple landing zones and selectable marker of the nucleic acid construct.

There are also provided methods for generating a recombinant microorganism containing multiple nucleic acids of interest from the resistance-pivot chassis-strain. In general the methods comprise transforming the resistance-pivot chassis-strain with a first nucleic acid insert comprising the nucleic acid of interest under conditions that allow recombination of the first nucleic acid insert with a first portion of nucleic acid construct in the genome of the microorganism. The methods then require a second transformation with a second nucleic acid insert under conditions that allow recombination of the second nucleic acid insert with a second portion of nucleic acid construct in the genome of the microorganism.

In one embodiment the first nucleic acid insert comprises a nucleic acid of interest (either alone or operably linked to a promoter) and a selectable marker that is different to the selectable marker in the resistance-pivot chassis strain. In this embodiment the first nucleic acid insert comprises, in a 5′ to 3′ direction the flanked by the first landing zone (e.g. LZA in FIG. 4), the nucleic acid of interest, the fifth landing zone (e.g. LZB2 in FIG. 4), the selectable marker, and the third landing zone (e.g. LZC1 in FIG. 4). The landing zones in the first nucleic acid insert can be at least 90% identical the corresponding landing zones in the resistance-pivot chassis strain.

Once the first nucleic acid insert is recombined into the genome of the resistance-pivot chassis strain, the strain comprises the first, fifth, third and fourth landing zones. That is the second landing zone is lost during recombination. In addition, the selectable marker is flanked by fifth and sixth landing zones.

The second nucleic acid insert comprises a second nucleic acid of interest (either alone or operably linked to a promoter) and a selectable marker that is different to the selectable marker in the resistance-pivot chassis (i.e. different to the selectable marker in the first nucleic acid insert. In this embodiment the second nucleic acid insert comprises, in a 5′ to 3′ direction the fifth landing zone (e.g. LZB2 in FIG. 4), the selectable marker, a sixth landing zone (e.g. LZC2 in FIG. 4), the second nucleic acid of interest, and the fourth landing zone (e.g. LZD in FIG. 4). The landing zones in the second nucleic acid insert can be at least 90% identical the corresponding landing zones in the resistance-pivot chassis strain, specifically the strain after recombination with the first nucleic acid insert.

Once the first and second nucleic acid inserts are recombined into the genome of the resistance-pivot chassis strain, the strain comprises the first, fifth, sixth and fourth landing zones in addition to a selectable marker and both the first and seconds nucleic acids of interest. The third landing zone is lost during recombination

In an alternate embodiment the first nucleic acid insert comprises a nucleic acid of interest (either alone or operably linked to a promoter) and a selectable marker that is different to the selectable marker in the resistance-pivot chassis strain. In this embodiment the first nucleic acid insert comprises, in a 5′ to 3′ direction the second landing zone (e.g. LZB1 in FIG. 4), the selectable marker, the sixth landing zone (e.g. LZC2 in FIG. 4), the nucleic acid of interest, and the fourth landing zone (e.g. LZD in FIG. 4). The landing zones in the first nucleic acid insert can be at least 90% identical the corresponding landing zones in the resistance-pivot chassis strain.

Once the first nucleic acid insert is recombined into the genome of the resistance-pivot chassis strain, the strain comprises the first, second, sixth, and fourth landing zones. That is the third landing zone is lost during recombination.

The second nucleic acid insert comprises a second nucleic acid of interest (either alone or operably linked to a promoter) and a selectable marker that is different to the selectable marker in the resistance-pivot chassis (i.e. different to the selectable marker in the first nucleic acid insert. In this embodiment the second nucleic acid insert comprises, in a 5′ to 3′ direction the first landing zone (e.g. LZA in FIG. 4), the second nucleic acid of interest, the fifth landing zone (e.g. LZB2 in FIG. 4), the selectable marker, and the sixth landing zone (e.g. LZC2 in FIG. 4). The landing zones in the second nucleic acid insert can be at least 90% identical the corresponding landing zones in the resistance-pivot chassis strain, specifically the strain after recombination with the first nucleic acid insert.

Once the first and second nucleic acid inserts are recombined into the genome of the resistance-pivot chassis strain, the strain comprises the first, sixth, fifth, and fourth landing zones in addition to a selectable marker and both the first and second nucleic acids of interest. The second landing zone is lost during recombination. In addition, the selectable marker is flanked by fifth and sixth landing zones.

In both of the preceding embodiments the recombinant microorganism comprises a selectable marker flanked by landing zones. Accordingly, further nucleic acid inserts can be designed and prepared to contain a different selectable marker, a further nucleic acid of interest and additional landing zones to facilitate yet further recombination events to include yet further nucleic acids of interest. Based on the teaching of this specification it is within the ability of a skilled person to design and prepare such further nucleic acid inserts using methods known in the art.

After each transformation step the methods require culturing the microorganism in the presence of a selection agent for the selectable marker in the nucleic acid insert to select for a recombinant microorganism comprising the nucleic acid of interest.

Additional confirmation that the nucleic acid insert or nucleic acid construct has been incorporated into the genome of the microorganism may be required in some instances. This may be achieved by any method known in the art, for example PCR, nucleic acid sequencing, southern blotting, RFLP analysis, and the like.

### Selectable Markers

Any selectable marker known in the art may be used in the nucleic acids and methods described herein.

The choice of selectable marker may vary depending on the cell or microorganism used. For example in embodiments where the cell is a microorganism a selectable marker may be a gene encoding antibiotic resistance.

Suitable selectable markers for use in cyanobacteria include genes that confer resistance to bleomycin, chloramphenicol, erythromycin, kanamycin, spectinomycin, neomycin, streptomycin, zeocin or gentamicin.

### Nucleic Acid Inserts

Any type of nucleic acid may be used as a nucleic acid insert, for example linear or circular DNA. The nucleic acid insert will contain a nucleic acid of interest, a number of landing zones and a selectable marker.

The nucleic acid of interest may be for example a sequence encoding a modified version of one or more cyanobacterial genes or may be one or more heterologous genes to be expressed in the cell.

The nucleic acid insert may be synthesised as a linear nucleic acid or may be constructed in a plasmid using molecular biology techniques known in the art. The plasmids may also contain replication origins for commonly used bacteria such as E. coli to facilitate modification of the nucleic acid insert sequences, and preparation of the plasmid containing the nucleic acid insert in an amendable species before transformation into a cell.

The nucleic acid insert can be isolated from a plasmid prior to use in the methods, for example the nucleic acid insert may be excised from a plasmid using restriction enzymes, or amplified by PCR, before use in the methods.

In some embodiments the nucleic acid insert comprises a promoter operatively coupled to the nucleic acid of interest.

A “promoter” refers to the DNA sequence(s) that control or otherwise modify transcription of a gene and can include binding sites for transcription factors, RNA polymerases, and other biomolecules and substances (e.g. inorganic compounds) that can influence transcription of a gene by interaction with the promoter. Typically these sequences are located at the 5′ end of the sense strand of the gene, but can be located anywhere in the genome.

As used herein, “operatively coupled” indicates that the regulatory sequences (e.g. the promoter) useful for expression of the coding sequences of a nucleic acid are placed in the nucleic acid molecule in the appropriate positions relative to the coding sequence so as to effect or enhance expression of the coding sequence. This same definition is sometimes applied to the arrangement of coding sequences and transcription control elements (e.g. promoters, enhancers, and termination elements), and/or selectable markers in an expression vector.

The promoter may be a constitutively active promoter or an inducible promoter. An inducible promoter is one that responds to a specific signal. In some embodiments an inducible promoter will not be activated in the absence of inducer, it will produce a predictable response to a given concentration of inducer or repressor. This response may be binary (i.e., on/off) or graded change with different concentrations of inducer/repressor. Ideally, saturating concentrations of the inducer is not harmful to the cyanobacteria host organism.

The inducible promoter may be a metal inducible promoter, a metabolite inducible promoter, a macronutrient inducible promoter.

The metal inducible promoter may be selected from the group comprising ArsB (induced by AsO²⁻), ziaA (induced by Cd²⁺ or Zn²⁺), coat (induced by Co²⁺ or Zn²⁺), nrsB (induced by Co²⁺ or Ni²⁺), petE (induced by Cu+²), isiAB (repressed by Fe³⁺), idiA (repressed by Fe²⁺), Smt (induced by Zn²⁺).

The metabolite inducible promoter may be selected from the group comprising the tetracycline inducible and the IPTG (Isopropyl β-D-1-thiogalactopyranoside) inducible tetR, trp-lac, Trc, A1lacO-1, trc10, trc20, LlacO1, clac143, and Trc. In one embodiment the inducible promoter is clac143.

The macronutrient inducible promoter may be selected from psbA2 (induced by light), psbA1 (induced by light), nirA (induced by NO₃⁻, repressed by NH₄⁺), and Nir (induced by NO₃⁻, repressed by NH₄⁻).

The promoter may be a Type I, Type II or Type III promoters. A type I promoter comprises transcriptional start site at +1 (by definition), a −10 element (consensus sequence 5′-TATAAT-3′), and a −35 element (consensus sequence 5′-TTGACA-3′). A type II promoter is usually used when expression of a gene is to be induced by stress or adaptation responses and thus are normally transcribed by group 2 sigma factors. Type II promoters have a −10 element but typically lack the −35 element. Type III promoters do not have regular −10 and −35 elements. Accordingly, the choice of promoter can be tailored to the desired growth conditions.

In some embodiments constitutive promoter may be used. Examples of suitable constitutive promoters include cpc560, psbA, plastocyanin promoter, BBaJ23101, and J23.

### Transformation Methods

Any transformation method known in the art may be used in the methods described herein. The choice or Transformation method will vary depending on the cell used in the methods and will be within the knowledge of the skilled person.

As used herein, the term “transformation” is used in the context of nucleic acid entering a cyanobacterial cell, to refer to the introduction of an exogenous and/or recombinant nucleic acid sequence into the interior of a living cyanobacteria. The nucleic acid may be in the form of naked DNA or RNA, it may be associated with various proteins or other elements such as lipids, or surfactants.

### Cyanobacteria

The nucleic acids and methods described herein are broadly applicable to any cyanobacteria capable of being transformed with a heterologous genetic element.

The cyanobacterial cells may be naturally competent. Alternatively, competence may be induced in the cell, for example using chemical, electrical or mechanical means or any other means known in the art.

Typically cyanobacterial cells are used in the methods when they are in an active growth phase. For example actively growing cyanobacteria are used in methods. The cyanobacteria may be in early, mid or late exponential phase. This can be determined using an OD measurement, for example at 750 nm. Cyanobacteria from a culture with an OD of 0.1 to 3.0 can be used. For example suitable ODs are 0.1, 0.2, 0.4, 0.6, 0.8, 1.0, 1.2, 1.4, 1.6, 1.8, 2.0, 2.2, 2.4, 2.6, 2.8, or 3.0.

Cyanobacteria cultured under any growth conditions known in the art can be used. In some embodiments the cyanobacteria are grown under low-light conditions, constant light or using periods of light and dark, for example light and dark periods that mimic a normal day/night cycle.

In embodiments where a light/dark cycle is used to prepare the cyanobacteria for transformation, the cyanobacteria may be harvested at any point in the light/dark cycle. However, it is known that in some (but not necessarily all) strains pilus biogenesis occurs daily in the morning, but natural competence is at is peak with the onset of darkness, that is natural cyanobacterial competence is conditional and tied to the cells' circadian rhythm. Accordingly, in some embodiments the cells are harvested at or near the transition from light to dark, or near the end of the light cycle.

The cyanobacteria cultured for transformation may be cultured in low-light conditions (i.e. less than 100 μmol photons·m⁻²·s⁻¹), for example 50 μmol photons·m⁻²·s⁻¹, normal light conditions (from 100-750 μmol photons·m⁻²·s⁻¹), for example 100-150 μmol photons·m⁻²·s⁻¹or light saturated conditions (greater than 750 μmol photons·m⁻²·s⁻¹). In embodiments where light/dark cycles are used the level of light in each light cycle may be independently selected from low-light, normal light or light saturated.

In some embodiments the cells are grown without controlling CO₂ levels. In other embodiments the cells are cultured in an atmosphere comprising about 0.05% to about 10% CO₂, for example the CO2 level is about 0.05%, 0.1%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5% or about 10%.

Most cyanobacteria harbor genes encoding proteins for type IV pili apparatus which are known to be involved in natural competence. Accordingly, it is envisaged that in some embodiments the methods disclosed herein can be used with any genus of cyanobacteria having type IV pili.

Cyanobacterial genera that can be used in the methods disclosed herein include those selected from the group comprising Collenia, Girvanella, Gunflintia, Morania, Sphaerocodium, Acaryochloris, Anabaena, Anabaenopsis, Aphanizomenon, Arthrospira, Aulosira, Borzia, Calothrix, Chlorogloeopsis, Chroococcidiopsis, Cyanobacterium, Cyanonephron, Cyanothece, Cylindrospermopsis, Cylindrospermum, Gloeobacter, Gloeocapsa, Gloeotrichia, Homoeothrix, Jakutophyton, Johannesbaptistia, Loefgrenia, Lyngbya, Merismopedia, Microcystis, Nodularia, Nostoc, Oscillatoria, Ozarkcollenia, Palaeolyngbya, Petalonema, Planktothrix, Prochlorococcus, Prochloron, Radaisia, Rivularia, Rothpletzella, Scytonema, Spirulina, Synechococcus, Synechocystis, Trichodesmium, and Wollea.

In some embodiments suitable strains include Synechocystis sp. PCC 6803, Synechococcus elongatus PCC 7942, Synechococcus sp. PCC 7002, Synechococcus sp.PCC 11901, Synechococcus sp. UTEX 2434, Synechococcus sp. UTEX 2973, Synechococcus sp. UTEX 3153, Synechococcus sp. UTEX 3154, Anabaena variabilis PCC 7120, and Leptolyngbya sp. BL0902

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### EXAMPLES

### Example 1: Universal Landing Zones and Marker-Enabled Flip

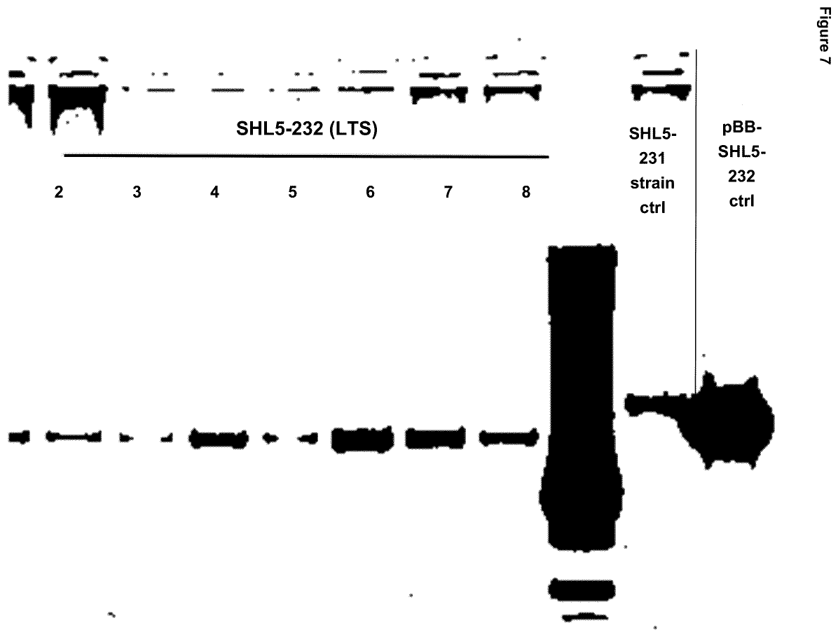

As exemplified herein, the inventors have demonstrated the use of Landing Zones and the exchange of the Selection Marker. This is detailed in FIG. 6(i) and experimental steps are described below, with an agarose gel detailing the change shown in FIG. 7.

### 1.1 Experimental Method—Strain Production

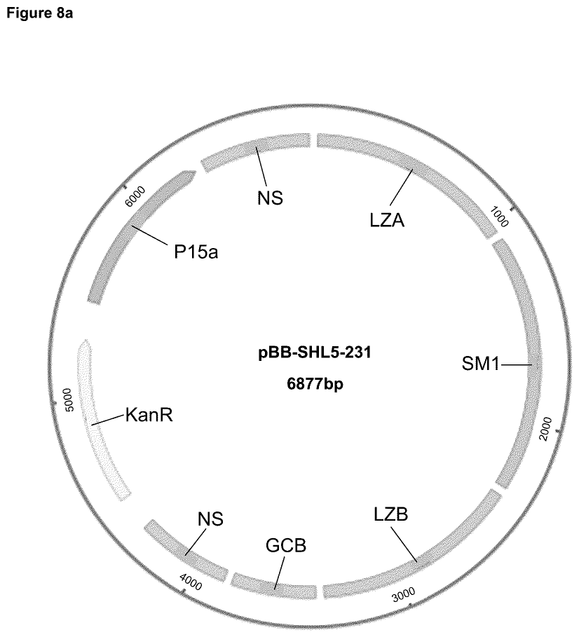

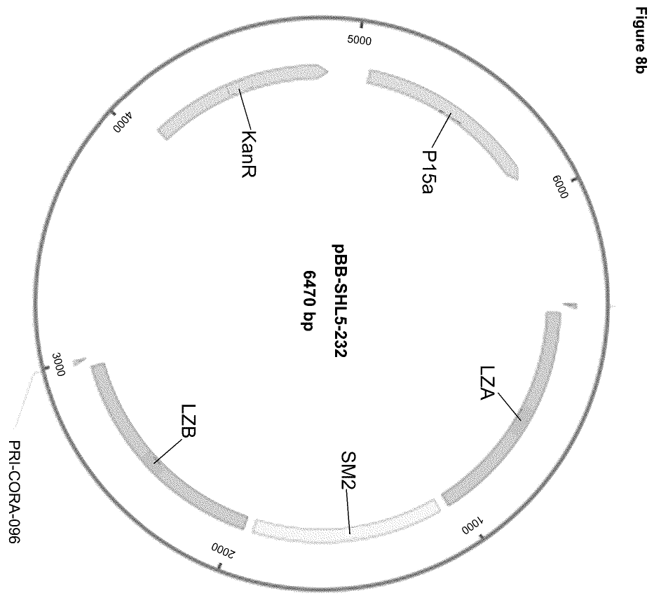

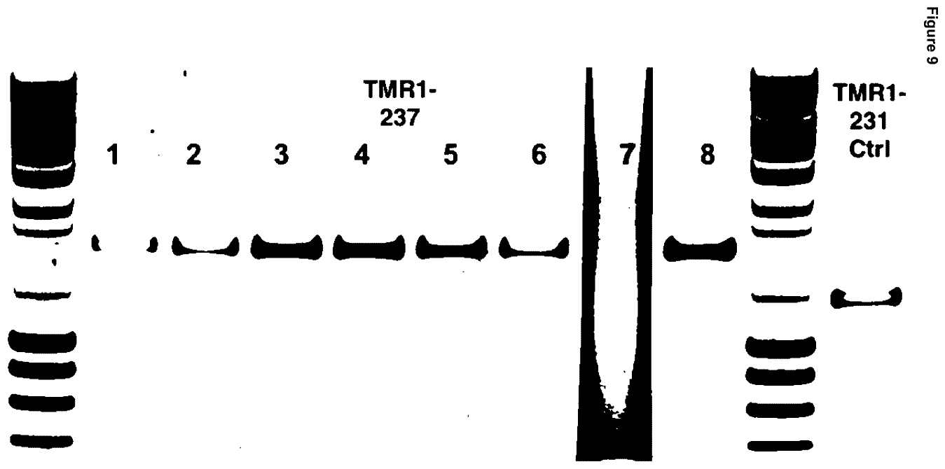

Synechococcus sp. PCC 7002 Wildtype (WT) cells were transformed with plasmid pBB-SHL5-231 (FIG. 8A). pBB-SHL5-231 comprises a portion of neutral site, landing zones, a selectable marker (SM1 which is a gene for spectinomycin resistance), a second landing zone and another portion if a neutral site. Transformants selected with spectinomycin (10 ug/mL) were designated strain SHL5-231.

Plasmid pBB-SHL5-232 (FIG. 8B) was used as a template for PCR amplification of a linear DNA (LIN-SHL5-001) using primers PRI-CORA-080 (5′-ACAGCCGTAGACTACAACGG-3′, SEQ ID NO: 18) and PRI-CORA-096 (5′-GTGTCACGTTACAGCTGCTA-3′, SEQ ID NO 19) pBB-SHL5-232 comprises a second selectable marker (SM2 which is a gene for gentamycin resistance) flanked by the same two landing zones in pBB-SHL5-231.

Strain SHL5-231 was grown in liquid medium supplemented with 20 ug/mL of Spectinomycin and washed with regular medium before the transformation to remove residual amounts of antibiotic.

Strain SHL5-231 was transformed with DNA LIN-SHL5-001 and transformants selected with gentamicin (10 ug/mL) were designated strain SHL5-232. Additionally, SHLS-232 colonies were screened by colony PCR. Given that SM2 is ˜500 bp shorter than SM1, a identifiable length shift can be observed on an agarose gel (FIG. 7), when compared to parent strain SHL5-231.

### 1.2 Experimental Method—Marker Enabled Flip

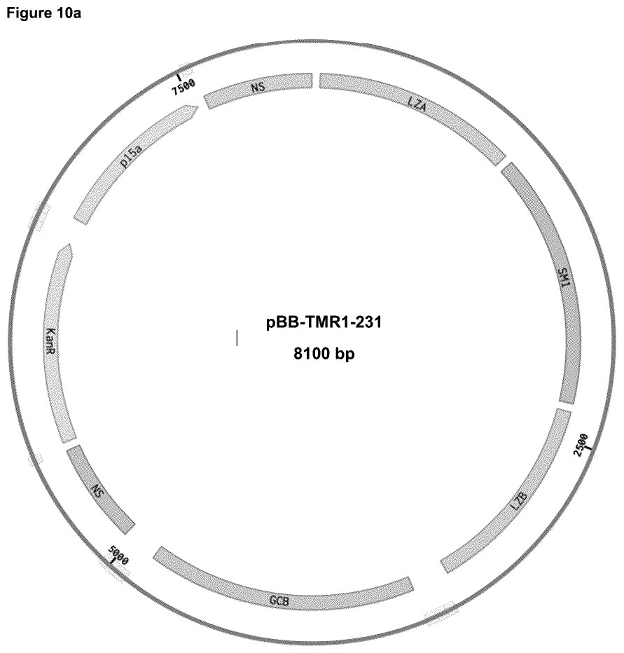

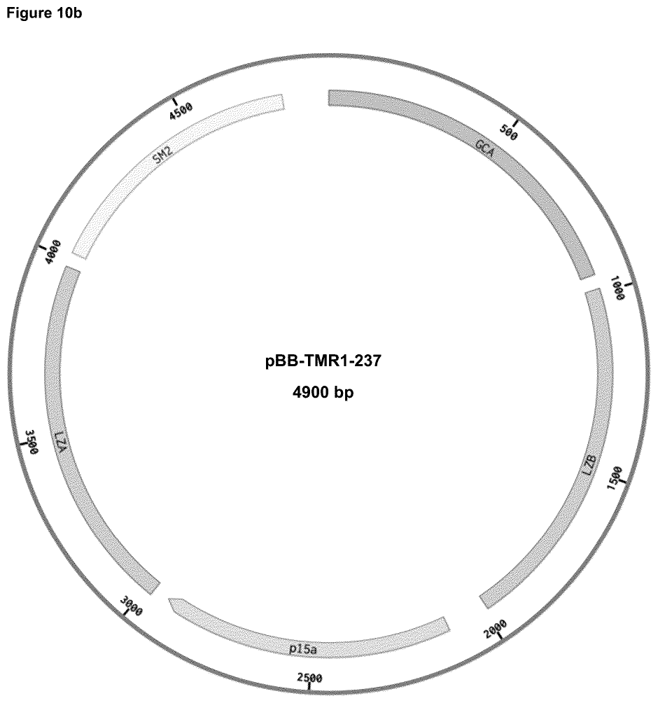

Step 1: PCC 7002 Wildtype (WT) cells were transformed with plasmid pBB-TMR1-231 (FIG. 10A), selected with spectinomycin (10 ug/mL), to generate cyanobacteria strain TMR1-231. LZA in FIGS. 6, 8 and 10 is SEQ ID NO: 1 and LZB is SEQ ID NO: 2

Step 2: Strain TMR1-231 was grown in liquid medium supplemented with 20 ug/mL of spectinomycin and washed with regular medium before the transformation (step 3) to remove residual amounts of antibiotic.

Step 3: Cyanobacteria strain TMR1-231 (step 1) was transformed with plasmid pBB-TMR1-237 (FIG. 10B), selected with gentamicin (10 ug/mL), to generate strain TMR1-237.

In addition to the ability for newly-engineered strains to grow on the alternative antibiotic, TMR1-237 colonies were also screened by colony PCR. Given that SM2+GCA (SEQ ID: 7) is ˜600 bp longer than SM1, an identifiable length shift can be observed on an agarose gel (FIG. 9), when compared to parent strain TMR1-231.

### Example 2: Landing Zones

The landing zones are artificial sequences designed to be amenable to homologous recombination. Landing zones are designed to mirror the GC content of native cyanobacterial DNA, while also not generating transcriptional output via the unintended incorporation of bacterial promoter sequences. Transcriptional terminators (TER) and translational insulators (TLT) can be included at either end of each Landing Zone cassette to avoid any potential readthrough to/from inserted Genetic Cassettes in engineered strains.

Landing Zones can be produced by randomly generating 1000 bp DNA sequences of approximately 50% GC content (50% is the average GC content in relevant cyanobacterial genomes).

Sequences are screened for potential bacterial promoters; where potential promoters were identified, sequences were altered until no potential promoters were found.

A collection of strong transcriptional terminators (TER) sequences was screened and repetitive parts removed.

A set of randomised translational insulators (TLT), including stop-codons in all x6 reading frames, were created and screened, removing repetitive parts.

A TLT and TER were added to both 5′ and 3′ ends of the 1000 bp DNA ‘core’ sequences. The ‘core’ sequence was then trimmed so that the complete LZ cassette (comprising 2 TLT, 2 TLT and a core) was approximately 1000 bp long.

The LZ cassettes were then screened for homology/interaction with each other and an internal parts database. TER/TLT sequences were interchanged between cassettes to ensure no significant repeats were present within the complete collection.

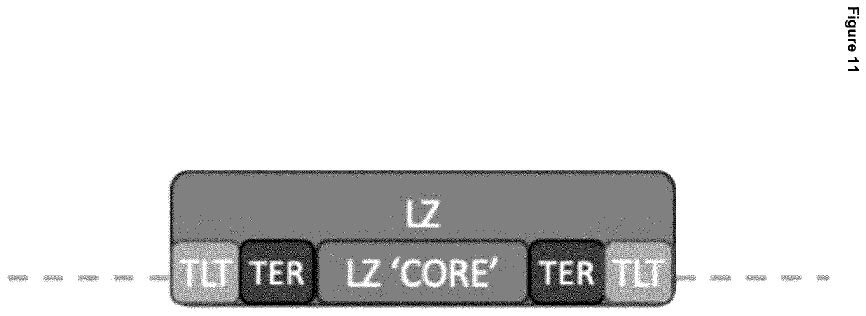

A schematic of a LZ is provided in FIG. 11.

Sequence Listing

| 1 | Sequence Listing | | |
|---|---|---|---|
| | Information | | |
| 1-1 | File Name | 1316440003US01.xml | |
| 1-2 | DTD Version | V1_3 | |
| 1-3 | Software Name | WIPO Sequence | |
| 1-4 | Software Version | 2.2.0 | |
| 1-5 | Production Date | 2023-03-23 | |
| 1-6 | Original free text | | |
| | language code | | |
| 1-7 | Non English free | | |
| | text language code | | |
| 2 | General | | |
| | Information | | |
| 2-1 | Current application: | | |
| | IP Office | | |
| 2-2 | Current application: | | |
| | Application number | | |
| 2-3 | Current application: | | |
| | Filing date | | |
| 2-4 | Current application: | 131644-0003US01 | |
| | Applicant file | | |
| | reference | | |
| 2-5 | Earliest priority | AU | |
| | application: IP | | |
| | Office | | |
| 2-6 | Earliest priority | 2020903460 | |
| | application: | | |
| | Application number | | |
| 2-7 | Earliest priority | 2020-09-25 | |
| | application: Filing | | |
| | date | | |
| 2-8en | Applicant name | Bondi Bio Pty Ltd | |
| 2-8 | Applicant name: | | |
| | Name Latin | | |
| 2-9 | Inventor name | | |
| 2-9 | Inventor name: | | |
| | Name Latin | | |
| 2-10en | Invention title | Standardised Cyanobacterial Strain Engineering | |
| 2-11 | Sequence Total | 19 | |
| | Quantity | | |
| 3-1 | Sequences | | |
| 3-1-1 | Sequence Number | 1 | |
| | [ID] | | |
| 3-1-2 | Molecule Type | DNA | |
| 3-1-3 | Length | 1000 | |
| 3-1-4-1 | Features | misc_feature 1..1000 | |
| | Location/Qualifiers | note = misc_feature | |
| | NonEnglishQualifier | | |
| | Value | | |
| 3-1-4-2 | Features | source 1..1000 | |
| | Location/Qualifiers | mol_type = other DNA | |
| | | organism = synthetic construct | |
| | NonEnglishQualifier | | |
| | Value | | |
| 3-1-5 | Residues | ggctagcagg gctcttccca ccacacgttt aaagcgcagt gcgaaccatg attccagcct | 60 |
| | | aagaaaggcg tggccgaaca taaggataag tcaaaagcgt gctagatcgt ttccggtacg | 120 |
| | | cgaaatggcg aaagctgttg gcactagctg agaggccctt agttcgcaga ggatgtgaga | 180 |
| | | gctgcggata cgggaacttc gcggacctga atacggagcg gaaatcaagt gcaggaattg | 240 |
| | | gatatccaaa gcctcatgga aaagcggcgg gactgctcct tggggatcga ggtaactacg | 300 |
| | | gactggcgcc ctactcaagg tacaggagag atgcatcgag cctgcataga gagctacaaa | 360 |
| | | acccacccat cgtcggacgt ttgccgcgct ccgcgagctt caacgcagtc atcaccaagg | 420 |
| | | ccgacgaagc agttttctcc gcgtagcagc ttgagatcaa caaatacgtt tggccctcca | 480 |
| | | caccaggaat gttgactgtg ttagatcaga tcagtgagtc ggaccggacg ggtacgtcct | 540 |
| | | ggctgtcttc agcagctatc cccttgccgt gggggtcgtg gactcgtgcg ttatcgagaa | 600 |
| | | cttgtgtcac ctcacatcga cccttgctga gaggcgcgag cgttacccag ccggttcatg | 660 |
| | | acctaccaac aaacctgtca tccgtaccgc ccatgccaaa tgtggctcca ggcgttcccg | 720 |
| | | aaggaattac taagcgttcc ggcggtctac gagatctaca gccaaatggg tgaaagaatt | 780 |
| | | tgcttgtttc ctcctgttac ctagcacaga tcgcagggcg gatggaccgc ttcttcaccg | 840 |
| | | tgcgataaag gaatcaccta gggctcgcgc tggtctcccg ccggcgttct gtcgccaggc | 900 |
| | | acgtaacaag gatgcgcagt ctacggccat ttcttgacgc tcgttgctca tgaggggcgt | 960 |
| | | cttcaaacgt gacgtgataa gttctaaagg tccagcgttg | 1000 |
| 3-2 | Sequences | | |
| 3-2-1 | Sequence Number | 2 | |
| | [ID] | | |
| 3-2-2 | Molecule Type | DNA | |
| 3-2-3 | Length | 1000 | |
| 3-2-4-1 | Features | misc_feature 1..1000 | |
| | Location/Qualifiers | note = misc feature | |
| | NonEnglishQualifier | | |
| | Value | | |
| 3-2-4-2 | Features | source 1..1000 | |
| | Location/Qualifiers | mol_type = other DNA | |
| | | organism = synthetic construct | |
| | NonEnglishQualifier | | |
| | Value | | |
| 3-2-5 | Residues | tatggtccgc aaggctgaac acggtggata tgcccgcaaa ccccagtgag gccagcccgg | 60 |
| | | cacggatggt ccgcaaacaa ttctgtagtt tgtgaagtag gctagtcttc cctggtaaga | 120 |
| | | cgatgcgttg ttagtgaggg catacgtgcg aatcagcgtt aacgagataa acgatgagca | 180 |
| | | atttgacgaa tgctcttctc aactctcttg gacatcgagg tgagtaagtg agcgacaacc | 240 |
| | | gaacggcggt cagcatgtct agagtcacgc acattgacta tccgttaatc acgtgtcgtt | 300 |
| | | tcggtaattc cggccactgc actgaatgtc tctgcaagcc cgttgtttcg aagtcctctc | 360 |
| | | atccattttg tgggagcgaa acagcgtttg cgcctgtcat atcactgcgg cctagcgatc | 420 |
| | | cgggcgcctg ttaggcatgt caaacggcta gttggtttgc tcgttgcgac tcaccgggtt | 480 |
| | | tcgttgtgaa caccgaggcc ggtgactagt tcactgtccc gctatgtgtt gacgatcatt | 540 |
| | | tccaagagga acgcttagca gctcggtttc cggcccggag tgactttttc ttgacggttg | 600 |
| | | gcggacgacg cctcctggtt cccacgctta cttgaatgtg ttgtgagaag gcgatggcga | 660 |
| | | ggacctgcct agtacagcgg ggactctctg gaaacccgca tttactgaag actgttccca | 720 |
| | | ggaacagcga tttctttctc acggtttaag agagaggctc tttgtcagct cctgtgaatg | 780 |
| | | ggcaaagcat cgcgggacaa ctggaagaca gacgtacccc cactcgcttc cctatgactc | 840 |
| | | tgaactcaaa agtttggctt tggtaagtcg tgtttgtgtg tcttgggtct ccaatgtccg | 900 |
| | | aattgggtcc gcacggggtc tttcatagat acacgggggc cgttggcccg actgcctctg | 960 |
| | | tttgtacctc gatattaaag gggttccgtt caagtcatgt | 1000 |
| 3-3 | Sequences | | |
| 3-3-1 | Sequence Number | 3 | |
| | [ID] | | |
| 3-3-2 | Molecule Type | DNA | |
| 3-3-3 | Length | 534 | |
| 3-3-4-1 | Features | source 1..534 | |
| | Location/Qualifiers | mol_type = other DNA | |
| | | organism = Synechococcus PCC7002 | |
| | NonEnglishQualifier | | |
| | Value | | |
| 3-3-5 | Residues | actgccgcac tcaatgttca accagatcta gtgattattt cggggttagc agccgatgcg | 60 |
| | | ggcaacctgg tgaagcaact gcgagaatta ggttacaacg gcattattgt tggggggaac | 120 |
| | | ggcttaaata cttctaatat tttccccgtc tgccaagcaa aatgtgatgg ggtgttggtg | 180 |
| | | gcccaagcct acagtgccga gttagataat gagattaacc gcgcgtttcg ggacgcctat | 240 |
| | | tttcaacaaa accaaaaaga gccgccccaa tttagtgccc aggcttttac ggcgatccaa | 300 |
| | | gtttttgttg aagccctcag cagcctcgat gaaaaaacgc ccttagaaac tcttgctcta | 360 |
| | | ccggacttgc gacgacaact gcgggacgaa atttttgcag gtacctacgt cacgcctttg | 420 |
| | | ggtgaaattt ccttcacaga ggaaggggaa attgtccaga aggaattttt tgtggcccaa | 480 |
| | | attgaaatgg atgaatcggg tcaacagggg cgtttcgcct tcattgaaac gaac | 534 |
| 3-4 | Sequences | | |
| 3-4-1 | Sequence Number | 4 | |
| | [ID] | | |
| 3-4-2 | Molecule Type | DNA | |
| 3-4-3 | Length | 457 | |
| 3-4-4-1 | Features | source 1..457 | |
| | Location/Qualifiers | mol_type = other DNA | |
| | | organism = Synechococcus PCC7002 | |
| | NonEnglishQualifier | | |
| | Value | | |
| 3-4-5 | Residues | ctagggagat tcttctaaag cgagcagggt ttgccaagtg gcgatcgcct cagggaacca | 60 |
| | | taaccaactt tgttcgaggc ggcgggggtt gagctgggaa cgatcgagat ctagggcgat | 120 |
| | | cgcccggagt ttttcggctt tccctacaag aatttcctgg tcaacgtcgg tgggttgcct | 180 |
| | | ttgggccaaa tcgtacatac taatcgccag gcccgcataa atctccgcct ggagggctgg | 240 |
| | | ggggaaatcc tgggtcgttg ccgccgctaa ggcttcatac caacgatcat tggcggcttc | 300 |
| | | gtaatctgca ttgaggtaat ggacaaaacc cagggccatc aaaatttcgg ggtcgttggg | 360 |
| | | ctgttgggct agggccatgt cccaattgtc ttgcaccgtg gcgatcgccg ttgcttgggt | 420 |
| | | tggcgatggt gggacggcag ccaaagttgt ttgccag | 457 |
| 3-5 | Sequences | | |
| 3-5-1 | Sequence Number | 5 | |
| | [ID] | | |
| 3-5-2 | Molecule Type | DNA | |
| 3-5-3 | Length | 487 | |
| 3-5-4-1 | Features | misc_feature 1..487 | |
| | Location/Qualifiers | note = misc_feature | |
| | NonEnglishQualifier | | |
| | Value | | |
| 3-5-4-2 | Features | source 1..487 | |
| | Location/Qualifiers | mol_type = other DNA | |
| | | organism = synthetic construct | |
| | NonEnglishQualifier | | |
| | Value | | |
| 3-5-5 | Residues | ggacctaacg ccttcagacc tagcagctgt aacgtgacac ggtgccacct gacgtctaag | 60 |
| | | aaaccattat tatcatgaca ttaacctttt gttatcaata aaaaaggccg cgatttgcgg | 120 |
| | | ccttattgtt cgtcttagtt agttagccct tagtgactcg aattcgcggc cgcttctaga | 180 |
| | | gtactagtag cggccgctgc aggagtcact aagggttagt tagttagcca attattgaag | 240 |
| | | acgcttaaca gcgtcttttt ttgtttctgg tctcccgctc actcaaaggc ggtaatctcg | 300 |
| | | agtcccgtca agtcagcgcg caataaaaaa gcccccggaa ggtgatcttc cgggggcttt | 360 |
| | | ctcatgcgtt ccaagttatg tatggaccgg ccgacagatc gtcaagatta ctataagatt | 420 |
| | | ccgcattgcg gacgatttag gcgcactatc gcgctcaagt tgatcgaatg tatgccccgg | 480 |
| | | atatgac | 487 |
| 3-6 | Sequences | | |
| 3-6-1 | Sequence Number | 6 | |
| | [ID] | | |
| 3-6-2 | Molecule Type | DNA | |
| 3-6-3 | Length | 1223 | |
| 3-6-4-1 | Features | misc_feature 1..1223 | |
| | Location/Qualifiers | note = misc_feature | |
| | NonEnglishQualifier | | |
| | Value | | |
| 3-6-4-2 | Features | source 1..1223 | |
| | Location/Qualifiers | mol_type = other DNA | |
| | | organism = synthetic construct | |
| | NonEnglishQualifier | | |
| | Value | | |
| 3-6-5 | Residues | gtgaaggcca gagttttgac agctagctca gtcctaggta taatgctagc tactagagta | 60 |
| | | gtggaggtta ctagatggtg aatgtgaaac cagtaacgtt atacgatgtc gcagagtatg | 120 |
| | | ccggtgtctc ttatcagacc gtttcccgcg tggtgaacca ggccagccac gtttctgcga | 180 |
| | | aaacgcggga aaaagtggaa gcggcgatgg cggagctgaa ttacattccc aaccgcgtgg | 240 |
| | | cacaacaact ggcgggcaaa cagtcgttgc tgattggcgt tgccacctcc agtctggccc | 300 |
| | | tgcacgcgcc gtcgcaaatt gtcgcggcga ttaaatctcg cgccgatcaa ctgggtgcca | 360 |
| | | gcgtggtggt gtcgatggta gaacgaagcg gcgtcgaagc ctgtaaagcg gcggtgcaca | 420 |
| | | atcttctcgc gcaacgcgtc agtgggctga tcattaacta tccgctggat gaccaggatg | 480 |
| | | ccattgctgt ggaagctgcc tgcactaatg ttccggcgtt atttcttgat gtctctgacc | 540 |
| | | agacacccat caacagtatt attttctccc atgaagacgg tacgcgactg ggcgtggagc | 600 |
| | | atctggtcgc attgggtcac cagcaaatcg cgctgttagc gggcccatta agttctgtct | 660 |
| | | cggcgcgtct gcgtctggct ggctggcata aatatctcac tcgcaatcaa attcagccga | 720 |
| | | tagcggaacg ggaaggcgac tttagtgcca tgtccggttt tcaacaaacc atgcaaatgc | 780 |
| | | tgaatgaggg catcgttccc actgcgatgc tggttgccaa cgatcagatg gcgctgggcg | 840 |
| | | caatgcgcgc cattaccgag tccgggctgc gcgttggtgc ggatatctcg gtagtgggat | 900 |
| | | acgacgatac cgaagacagc tcatgttata tcccgccgtt aaccaccatc aaacaggatt | 960 |
| | | ttcgcctgct ggggcaaacc agcgtggacc gcttgctgca actctctcag ggccaggcgg | 1020 |
| | | tgaagggcaa tcagctgttg cccgtctcac tggtgaaaag aaaaaccacc ctggcgccca | 1080 |
| | | atacgcaaac cgcctctccc cgcgcgttgg ccgattcatt aatgcagctg gcacgacagg | 1140 |
| | | tttcccgact ggaaagcggg cagtaactcg gtaccaaatt ccagaaaaga gacgctgaaa | 1200 |
| | | agcgtctttt ttcgttttgg tcc | 1223 |
| 3-7 | Sequences | | |
| 3-7-1 | Sequence Number | 7 | |
| | [ID] | | |
| 3-7-2 | Molecule Type | DNA | |
| 3-7-3 | Length | 878 | |
| 3-7-4-1 | Features | misc_feature 1..878 | |
| | Location/Qualifiers | note = misc feature | |
| | NonEnglishQualifier | | |
| | Value | | |
| 3-7-4-2 | Features | source 1..878 | |
| | Location/Qualifiers | mol_type = other DNA | |
| | NonEnglishQualifier | organism = synthetic construct | |
| | Value | | |
| 3-7-5 | Residues | ttaacaagat gtaattgaca taagtcccat caccgttgta taaatgtgtg gaattgtgag | 60 |
| | | cggataacaa tttcacacat actagagtag tggaggttac tagatggtga gcaagggcga | 120 |
| | | ggagctgttc accggggtgg tgcccatcct ggtcgagctg gacggcgacg taaacggcca | 180 |
| | | caagttcagc gtgtccggcg agggcgaggg cgatgccacc tacggcaagc tgaccctgaa | 240 |
| | | gctgatctgc accaccggca agctgcccgt gccctggccc accctcgtga ccaccctggg | 300 |
| | | ctacggcgtg cagtgcttcg cccgctaccc cgaccacatg aagcagcacg acttcttcaa | 360 |
| | | gtccgccatg cccgaaggct acgtccagga gcgcaccatc ttcttcaagg acgacggcaa | 420 |
| | | ctacaagacc cgcgccgagg tgaagttcga gggcgacacc ctggtgaacc gcatcgagct | 480 |
| | | gaagggcatc gacttcaagg aggacggcaa catcctgggg cacaagctgg agtacaacta | 540 |
| | | caacagccac aacgtctata tcaccgccga caagcagaag aacggcatca aggccaactt | 600 |
| | | caagatccgc cacaacatcg aggacggcgg cgtgcagctc gccgaccact accagcagaa | 660 |
| | | cacccccatc ggcgacggcc ccgtgctgct gcccgacaac cactacctga gctaccagtc | 720 |
| | | caagctgagc aaagacccca acgagaagcg cgatcacatg gtcctgctgg agttcgtgac | 780 |
| | | cgccgccggg atcactctcg gcatggacga gctgtacaag taactcggta ccaaattcca | 840 |
| | | gaaaagacac ccgaaagggt gttttttcgt tttggtcc | 878 |
| 3-8 | Sequences | | |
| 3-8-1 | Sequence Number | 8 | |
| | [ID] | | |
| 3-8-2 | Molecule Type | DNA | |
| 3-8-3 | Length | 893 | |
| 3-8-4-1 | Features | source 1..893 | |
| | Location/Qualifiers | mol_type = other DNA | |
| | | organism = Synechococcus PCC7942 | |
| | NonEnglishQualifier | | |
| | Value | | |
| 3-8-5 | Residues | gatccggcag ccggcggagc gctgctttct tggcaagcgg tcgccagccc caacgccagg | 60 |
| | | gctgccagcc cgaaacagcg gggcaaggca gcttggaagg gcgatcgcag cacgggcatg | 120 |
| | | gcaatgtctc tctgaaggaa tgcagacctt attcgtacag ccagggttga atcgtggggg | 180 |
| | | tccaatcact tagctctgct gggctaaacc agagagcaat ttcctgttgt gctgtttcga | 240 |
| | | ttgcatccga gccatggatg atgttgcggc caatattgac accaaaatca ccacggatgg | 300 |
| | | tgcccggttc tgccgtcagc ggattggtag cgccgatcaa cttgcgagca gccgccacaa | 360 |
| | | cgccttcgcc ttccaagacg atcgccacga tcggcccaga ggtgatgaac tcgacgaggc | 420 |
| | | cattgaagaa ggggcgctcg cggtggacag catagtgctg ttcggccagc tcgcgactgg | 480 |
| | | gcttcagctg ctttaggccc accagtttga agcctttttg ctcaaagcgg ccgatgatcg | 540 |
| | | taccgaccaa accccgctga acgccatcgg gcttgatggc aataaatgtg cgttccacag | 600 |
| | | acatctagat agtcctcaag acgaggcaag cattgagctt gccttcctat ggttcgggat | 660 |
| | | cactgggatt cttgacaagc gatcgcggtc acatcgctat ctcttaggac ttcgcagcgg | 720 |
| | | gcgagtcgga ttgacccggt agggatttcg ccagatcaat gcccgtggtt tgtttcagct | 780 |
| | | tctccagcaa gctagcgatt tgggtagcgc tgccttcccc ttcgccaatc acagtgatcg | 840 |
| | | actccacgtc gatatctggc acggtgcctg aaagcgtgac gagcagggac tcg | 893 |
| 3-9 | Sequences | | |
| 3-9-1 | Sequence Number | 9 | |
| | [ID] | | |
| 3-9-2 | Molecule Type | DNA | |
| 3-9-3 | Length | 886 | |
| 3-9-4-1 | Features | source 1..886 | |
| | Location/Qualifiers | mol_type = other DNA | |
| | | organism = Synechococcus PCC7942 | |
| | NonEnglishQualifier | | |
| | Value | | |
| 3-9-5 | Residues | ttccgcgatc gcccgtttac aagctgcctc agctggggcg atcacatcgg cttgaagttg | 60 |
| | | ctgctgcacc tgtttgatcc gctcctgctg cacagggagt tctgcttggc tacgagcgac | 120 |
| | | ttcggtagca atgtccgctt cagcttcggc caccaccgct tcgcgccgcg tcaacgcatc | 180 |
| | | ctgaatccgg cgctcggcct cggcttgggc gatcgctaca tcgcgatcga tccgacgcag | 240 |
| | | ggccgtgatc ttgtcatttt cggccgtttg gatcgcagag gcagcctggg catcggcttc | 300 |
| | | agcaattcgg gcatctcgct gcagatcagc ccgctgcttg cgtccactag ccgagagata | 360 |
| | | accgacctca tcggaaatgt tctggacttg cagcgtatcg aggactagac ccagctgctc | 420 |
| | | aaggtcatcc tccgcctctt ccagcagact tttggcaaag gcaattttgt cctcgttgat | 480 |
| | | ctgctccggc gtgaggctgg ctaaaacacc acgcaagttg ccttcgaggg tctccttggc | 540 |
| | | aatttgctcg atttccttac ggtttttgcc aagcagccgc tcgatcgcgt tgtggatggt | 600 |
| | | cggttcttcc ccagcaatct tgatattggc aacgccttca acagtcaggg gaatgccgcc | 660 |
| | | cttggagaag gcattggaaa cgcgcaactc aatgatcatg ttggtcagat ccatgcggag | 720 |
| | | cgctttttcc agcagaggta cccgcaggct gctgccgccc ttgaccaagc gatagccaac | 780 |
| | | tcggcggcca tcactactgc ggcgactact gccagcaaag atcaaaattt cactgggttg | 840 |
| | | gcagatgtag tagagattgc gcaggactaa gctgccagcc ccggcg | 886 |
| 3-10 | Sequences | | |
| 3-10-1 | Sequence Number | 10 | |
| | [ID] | | |
| 3-10-2 | Molecule Type | DNA | |
| 3-10-3 | Length | 798 | |
| 3-10-4-1 | Features | source 1..798 | |
| | Location/Qualifiers | mol_type = other DNA | |
| | | organism = Synechococcus PCC7942 | |
| | NonEnglishQualifier | | |
| | Value | | |
| 3-10-5 | Residues | cgtctgcaag aagccggtgc ccatctcatt tttgacgata tgcgactgct gcccagtctg | 60 |
| | | ctccaatcgt ccccaaaaga taactccaca gcattgccca atccctaacc cctgctcgcg | 120 |
| | | ccgcaactac acactaaacc gttcctgcgc gatcgctctt actgttgatg gctcgtgctt | 180 |
| | | aaaaacaatg caaccctaac cgtttcagct ggtgattttc ggacgatttg gcttacaggg | 240 |
| | | ataactgaga gtcaacagcc tctgtccgtc attgcacacc catccatgca ctggggactt | 300 |
| | | gactcatgct gaatcacatt tcccttgtcc attgggcgag aggggagggg aatcttctgg | 360 |
| | | actcttcact aagcggcgat cgcaggttct tctacccaag cagtggcgat cgcttgattg | 420 |
| | | cagtcttcaa tgctggcctc tgcagccatc gccgccacca aagcatcgta ggcgggacgt | 480 |
| | | tgttgctcca gtaaagtctt cgcccgtaac aatccccagc gactgcgtaa atccgcttcg | 540 |
| | | gcaggattgc gatcgagttg ccgccacagt tgtttccact gggcgcgatc gtcagctccc | 600 |
| | | ccttccacgt tgccgtagac cagttgctct gccgctgcac cggccatcaa cacctgacac | 660 |
| | | cactgttcca gcgatcgctg actgagttgc ccctgtgcgg cttcggcttc tagcgcagct | 720 |
| | | gcttggaact gcacaccccc gcgaccaggt tgtccttggc gcagcgcttc ccacgctgag | 780 |
| | | agggtgtagc ccgtcacg | 798 |
| 3-11 | Sequences | | |
| 3-11-1 | Sequence Number | 11 | |
| | [ID] | | |
| 3-11-2 | Molecule Type | DNA | |
| 3-11-3 | Length | 733 | |
| 3-11-4-1 | Features | source 1..733 | |
| | Location/Qualifiers | mol_type = other DNA | |
| | NonEnglishQualifier | organism = Synechococcus PCC7942 | |
| | Value | | |
| 3-11-5 | Residues | ggtaacccca gcgcggttgc taccaagtag tgacccgctt cgtgatgcaa aatccgctga | 60 |
| | | cgatattcgg gcgatcgctg ctgaatgcca tcgagcagta acgtggcacc ccgcccctgc | 120 |
| | | caagtcaccg catccagact gaacagcacc aagaggctaa aacccaatcc cgccggtagc | 180 |
| | | agcggagaac tacccagcat tggtcccacc aaagctaatg ccgtcgtggt aaaaatcgcg | 240 |
| | | atcgccgtca gactcaagcc cagttcgctc atgcttcctc atctaggtca cagtcttcgg | 300 |
| | | cgatcgcatc gatctgatgc tgcagcaagc gttttccata ccggcgatcg cgccgtcgcc | 360 |
| | | ctttcgctgc cgtggcccgc ttacgagctc gtttatcgac cacgatcgca tccaaatccg | 420 |
| | | cgatcgcttc ccagtccggc aattcagtct ggggcgtccg tttcattaat cctgatcagg | 480 |
| | | cacgaaattg ctgtgcgtag tatcgcgcat agcggccagc ctctgccaac agcgcatcgt | 540 |
| | | gattgcctgc ctcaacaatc tggccgcgct ccatcaccaa gatgcggctg gcattacgaa | 600 |
| | | ccgtagccag acggtgagca atgataaaga ccgtccgtcc ctgcatcacc cgttctaggg | 660 |
| | | cctcttgcac caaggtttcg gactcggaat caagcgccga agtcgcctca tccagaatta | 720 |
| | | aaatgcgtgg atc | 733 |
| 3-12 | Sequences | | |
| 3-12-1 | Sequence Number | 12 | |
| | [ID] | | |
| 3-12-2 | Molecule Type | DNA | |
| 3-12-3 | Length | 798 | |
| 3-12-4-1 | Features | source 1..798 | |
| | Location/Qualifiers | mol_type = other DNA | |
| | | organism = Synechococcus PCC7002 | |
| | NonEnglishQualifier | | |
| | Value | | |
| 3-12-5 | Residues | ccgattagac cctaaatttc cagaattttg cctatttcgt ctgattaata aggtaattgg | 60 |
| | | gaataggcag cctgggaaat agtcggcaac tccgcatatt tgccgaggat cgattgcacc | 120 |
| | | attgcataga gacaagccac taaagtaccg agaaaggcaa catttgaaag ggtttcaacg | 180 |
| | | agaagactgc cgcccccaaa gccaaaaaag cgaattaaaa tcccgaacaa caccagggca | 240 |
| | | atgtcaatca aaatcgcctg gagggtattg aagcgaatga agtggctaat tttgctgttg | 300 |
| | | cgtaccaccg cagcatagag aatgaagaaa ataatcagcc ctgcaaaggg aaatgaataa | 360 |
| | | aggcggatta ggggttgtaa aggcacatag attaccccca ggatcggaaa gcgcattaaa | 420 |
| | | aacggcagac cgaagggcaa agcgtagacg aggggtaata ggtaaggtaa ggccccaaat | 480 |
| | | aaacgatctg tgggttcagt ggtagtggac ataaaacttt ttctgcaatg gactagctcc | 540 |
| | | tattctacag agaagtcttg aagaagttct gtggcgatcg ccagggccaa acaatgggaa | 600 |
| | | aaattaaacg aaattcagga attttgcggc gatttcccct gcatcctgac aaaaggcagt | 660 |
| | | agaattgtaa acatttgtta atctcttttt gaaactgaat gcaaagtaca gtccgctccc | 720 |
| | | cagggagtcg cgagtctcta cggcaagatc tgcccttcac cctcaaggat gtgaaagcag | 780 |
| | | ccatcccaga ctactgtt | 798 |
| 3-13 | Sequences | | |
| 3-13-1 | Sequence Number | 13 | |
| | [ID] | | |
| 3-13-2 | Molecule Type | DNA | |
| 3-13-3 | Length | 809 | |
| 3-13-4-1 | Features | source 1..809 | |
| | Location/Qualifiers | mol_type = other DNA | |
| | | organism = Synechococcus PCC7002 | |
| | NonEnglishQualifier | | |
| | Value | | |
| 3-13-5 | Residues | gagccatgct cccatttggc ggagtttctt tagatcccaa aaggcttgtc actacatcgc | 60 |
| | | cgatcagggt tctcacttgt attaccagcc gaaaaaataa gctccaaaag cgtgactaga | 120 |
| | | tagcgctgta aaccctaatt ttcctcggag aattggggtt tttctttggt tggtgatgat | 180 |
| | | gatttttaga gagatcgcct atgatcgaga ccgttgaatt tatggcagca acatgaaacg | 240 |
| | | ggttcgggtc aagtacagtt caccagtctt ttggctgggt cttggtttaa ccggcatttt | 300 |
| | | gggcattggg gcgatcgcca accgcaatgc tttacttccc ttgtttccag gcagtggtca | 360 |
| | | ggtggaatct ccagcagcgg tagattctga agtgttagcc ctggtggatc tcgccccggc | 420 |
| | | agagcggcgc gatcgcctcg aagtgatcgc caacagcagc aacaatgacc tcgaccgaaa | 480 |
| | | ccgcgcccgt tacctgttgg ggatggacta cttggttgcg gaagatgggg cagcggccct | 540 |
| | | agccgccttc gagaacctag aacaggacta tccggtgtta acgcctcata ttttgattaa | 600 |
| | | acggggccgc gcctacgaat tggtcaataa tcccgaacag gcccaggtga tttggtttga | 660 |
| | | tgtggtgcaa aattatccag aggatgctgc cgctgccgaa gcgctgtttc gcctcagtgc | 720 |
| | | ctacgacccg aaatatgccg accaggcgat cgccgaatat cccgcccatc cgcgcaccca | 780 |
| | | aagcttgatc caacaacgcc ttgcagaaa | 809 |
| 3-14 | Sequences | | |
| 3-14-1 | Sequence Number | 14 | |
| | [ID] | | |
| 3-14-2 | Molecule Type | DNA | |
| 3-14-3 | Length | 623 | |
| 3-14-4-1 | Features | source 1..623 | |
| | Location/Qualifiers | mol_type = other DNA | |
| | | organism = Synechocystis PCC6803 | |
| | NonEnglishQualifier | | |
| | Value | | |
| 3-14-5 | Residues | ggcaatgccc actcctccac aggcggtgag ggtaaagctc agcagtaaag ttaccaggac | 60 |
| | | tacagcagtg gatagggact ttttcaacat gggagaaagg gaagaactgg gcaagaaggc | 120 |
| | | aaaattacct ttccttaccc attaaacctc caatggttga ccagaaacta gaggtagaat | 180 |
| | | gattcccgcc agaaaaagaa gtttaacaat ttgccatggg cactgttctg aagcggggag | 240 |
| | | gaaggctatg gcctcctcaa ccaatttatt tcctgcctaa cccaggggac gttgttgagg | 300 |
| | | ctataagttg aggctataaa tttaacttat taaaaggttc gacaaatttg agatagtttt | 360 |
| | | gtggcaaaga tactgcttag gaaccaaata ttgcataaac ttagagatat agttttttct | 420 |
| | | aaaaaaatag tcttatttct atctattgaa tcggggcaat ttaaactcag aatagattag | 480 |
| | | ttgttcccag ctgaaaccat cgtgtgcttt ttccagaggc gtttttggca atttttcctc | 540 |
| | | tggtaaattt caccgacttt ggggcaatgc tcataatcac catagagtga aatccatgaa | 600 |
| | | caagtttgaa tcaagacaat cgg | 623 |
| 3-15 | Sequences | | |
| 3-15-1 | Sequence Number | 15 | |
| | [ID] | | |
| 3-15-2 | Molecule Type | DNA | |
| 3-15-3 | Length | 587 | |
| 3-15-4-1 | Features | source 1..587 | |
| | Location/Qualifiers | mol_type = other DNA | |
| | | organism = Synechocystis PCC6803 | |
| | NonEnglishQualifier | | |
| | Value | | |
| 3-15-5 | Residues | gctctttttc ctgggttgtg gtgtcggctt actactgtcg gtggtttggg tcaatgttgc | 60 |
| | | tcgccatagt cctccgctag aatcctcccc agtcaaggtc tcgcctccct tccaggtcga | 120 |
| | | ctagtcacaa caatttaaaa atcagaaaaa ttgtcccatt gatcaactta cagggggcca | 180 |
| | | ttgagcaaaa tccggggtca ccatctagtc cccaaaaagc tggcgatcgc caaataatag | 240 |
| | | taaaacttat cattcaaatt taaaattact tagcagatcc agggggacaa ctgcaaaatt | 300 |
| | | ggtcggattt acatatagac tttagcttat agatttcaag acataggcat tcaaacctgc | 360 |
| | | atagacaaga gtctatacag agcgaagcca atggggttca ttgcccctgg aaagatcaag | 420 |
| | | caaactgccg aagattcagg gccaagcttt actaccccaa tccccataaa tttcaaccaa | 480 |
| | | ggagacaatt tacattatgg attttttgtc caatttcttg acggacttcg tgggacaatt | 540 |
| | | gcagtcccca accctagcct ttctgattgg ggggatggtt attgccg | 587 |
| 3-16 | Sequences | | |
| 3-16-1 | Sequence Number | 16 | |
| | [ID] | | |
| 3-16-2 | Molecule Type | DNA | |
| 3-16-3 | Length | 501 | |
| 3-16-4-1 | Features | source 1..501 | |
| | Location/Qualifiers | mol_type = other DNA | |
| | | organism = Synechocystis PCC6803 | |
| | NonEnglishQualifier | | |
| | Value | | |
| 3-16-5 | Residues | atgactattc aatacacccc cctagccgat cgcctgttgg cctacctcgc cgccgatcgc | 60 |
| | | ctaaatctca gcgccaagag tagttccctc aacaccagta ttctgctcag cagtgaccta | 120 |
| | | ttcaatcagg aagggggaat tgtaacagcc aactatggct ttgatggtta tatgggaatt | 180 |
| | | cccggtatgg atggcaccga tgcggaatcc caacagattg cctttgacaa caatgtggcc | 240 |
| | | tggaataacc tgggggattt gtccaccacc acccaacggg cctacacttc ggctattagc | 300 |
| | | acagacacag tgcagagtgt ttatggcgtt aatctggaaa aaaacgataa cattcccatt | 360 |
| | | gtttttgcgt ggcccatttt tcccaccacc cttaatccca cagattttca ggtaatgctt | 420 |
| | | aacacggggg aaattgtcac cccggtgatc gcctctttga ttcccaacag tgaatacaac | 480 |
| | | gaacggcaaa cggtagtaat t | 501 |
| 3-17 | Sequences | | |
| 3-17-1 | Sequence Number | 17 | |
| | [ID] | | |
| 3-17-2 | Molecule Type | DNA | |
| 3-17-3 | Length | 522 | |
| 3-17-4-1 | Features | source 1..522 | |
| | Location/Qualifiers | mol_type = other DNA | |
| | | organism = Synechocystis PCC6803 | |
| | NonEnglishQualifier | | |
| | Value | | |
| 3-17-5 | Residues | acccagatgg gaaggtattt tatgcttcct ttgctgccgc tgatgaccaa gccacggatt | 60 |
| | | taaccacggc gatcgccaat cccacggcca tcgatttaat taacgccagg ggatttacgg | 120 |
| | | cgggtagttc cgtcaccgta tcgggttcct acagtcggga agcctttttt gatggatcca | 180 |
| | | tgggttttta tcgacttctg gacgataacg gtgcagtgct agatccctta acaggtggtg | 240 |
| | | taatcaaccc aggacaggta ggttatcaag aagcagcttt ggcagatagc aatcgtttgc | 300 |
| | | aagccactgg ctccacccta acggcagaag acctagaaac cagagcattt tccttcaata | 360 |
| | | ttttgggtgg cgagttgtat gcgccatttt taacggttaa tgacagtctt tccggtatta | 420 |
| | | atcagactta ttttgccttt gggtcggcca acccagatgg catcagccac agcacaaact | 480 |
| | | tgggacccaa cgtgattggt tttgaagatt ttctcggcgg ag | 522 |
| 3-18 | Sequences | | |
| 3-18-1 | Sequence Number | 18 | |
| | [ID] | | |
| 3-18-2 | Molecule Type | DNA | |
| 3-18-3 | Length | 20 | |
| 3-18-4-1 | Features | misc_feature 1..20 | |
| | Location/Qualifiers | note = primer_bind | |
| | NonEnglishQualifier | | |
| | Value | | |
| 3-18-4-2 | Features | source 1..20 | |
| | Location/Qualifiers | mol_type = other DNA | |
| | NonEnglishQualifier | organism = synthetic construct | |
| | Value | | |
| 3-18-5 | Residues | acagccgtag actacaacgg | 20 |
| 3-19 | Sequences | | |
| 3-19-1 | Sequence Number | 19 | |
| | [ID] | | |
| 3-19-2 | Molecule Type | DNA | |
| 3-19-3 | Length | 20 | |
| 3-19-4-1 | Features | misc_feature 1..20 | |
| | Location/Qualifiers | note = primer_bind | |
| | NonEnglishQualifier | | |
| | Value | | |
| 3-19-4-2 | Features | source 1..20 | |
| | Location/Qualifiers | mol_type = other DNA | |
| | | organism = synthetic construct | |
| | NonEnglishQualifier | | |
| | Value | | |
| 3-19-5 | Residues | gtgtcacgtt acagctgcta 20 | 20 |

1. A nucleic acid cassette comprising;
two portions of a neutral site sequence; and
a heterologous nucleic acid comprising at least two landing zones and a first selectable marker gene located between the landing zones; and
wherein the heterologous nucleic acid is between the two portions of the neutral site; and
wherein the neutral site sequence is substantially homologous to at least a part of a non-essential region of Cyanobacteria.

2. The nucleic acid cassette of claim 1, wherein the Cyanobacteria is selected from the group consisting of Synechocystis sp. PCC 6803, Synechococcus elongatus PCC 7942, Synechococcus sp. PCC 7002, Synechococcus sp. PCC 11901, Synechococcus sp. UTEX 2434, Synechococcus sp. UTEX 2973, Synechococcus sp. UTEX 3153, Synechococcus sp. UTEX 3154, Anabaena variabilis PCC 7120, and Leptolyngbya sp. BL0902.

3. The nucleic acid cassette of claim 1, wherein the non-essential region is selected from the NSC1 (GenBank QWO81945) region of Synechocystis sp. strain PCC 6803, the slr0168 (GenBank QWO79510) region of Synechocystis sp. strain PCC 6803, the A0159 (GenBank BAW95305) region of Synechococcus sp. strain PCC 7002, the A2842 (GenBank ACA99827) region of Synechococcus sp. strain PCC 7002, the NS1 (GenBank AAA81020), NS2 (GenBank AAA86649) or a non-essential region of Synechococcus sp. strain PCC 7942.

4. The nucleic acid cassette of claim 1, wherein the length of each neutral site sequence portion is independently selected from about 500 bp, about 550 bp, about 600 bp, about 650 bp, about 700 bp, about 750 bp, about 800 bp, about 850 bp, about 900 bp, about 950 bp, or at least about 1000 bp.

5. The nucleic acid cassette of claim 1, wherein the landing zone comprises a core sequence consisting of a randomly generated nucleic acid sequence with a GC content of approximately 50% and lacking a bacterial promoter sequence.

6. The nucleic acid cassette of claim 5, wherein the landing zone further comprises at least one transcriptional terminator and at least one translational insulator, preferably the landing zone comprises a transcriptional terminator and a translational insulator at either end of the core sequence.

7. The nucleic acid cassette of claim 6, wherein the landing zone comprises SEQ ID NO: 1 or SEQ ID NO: 2.

8. The nucleic acid cassette of claim 1, wherein the neutral site portion may be selected from a portion of any one of SEQ ID NOs: 8-17.

9. The nucleic acid cassette of claim 1, wherein the neutral site portions are SEQ ID NO: 3 and SEQ ID NO: 4.

10. The nucleic acid cassette of claim 1, wherein each landing zone is the same or different.

11. The nucleic acid cassette of claim 1, wherein the selectable marker gene is selected from genes that confer resistance to bleomycin, chloramphenicol, erythromycin, kanamycin, spectinomycin, neomycin, streptomycin, zeocin or gentamicin.

12. A Cyanobacterium cell comprising the nucleic acid cassette of claim 1.

13. The Cyanobacterium cell of claim 12, wherein the nucleic acid cassette is integrated into the genome of the Cyanobacterium cell.

14. The nucleic acid cassette of claim 1, wherein the heterologous nucleic acid comprises, in a 5′ to 3′ direction a first landing zone, a second landing zone, a first selectable marker, a third landing zone, and a fourth landing zone.

15. A Cyanobacterium cell comprising the nucleic acid cassette of claim 14.

16. The Cyanobacterium cell of claim 15, wherein the nucleic acid cassette is integrated into the genome of the Cyanobacterium cell.

17. A method for generating a recombinant Cyanobacterium cell comprising a nucleic acid of interest, the method comprising:
contacting the cell of claim 13 with a nucleic acid insert under conditions that allow recombination of the nucleic acid insert with the nucleic acid cassette in the genome of the cell; wherein
the nucleic acid insert comprises the nucleic acid of interest and a second selectable marker flanked by two landing zones, each landing zone comprising a sequence at least 90% identical to the landing zones in the cell's genome.

18. The method of claim 17, further comprising culturing the cell in the presence of a selection agent for the second selectable marker, thereby selecting a recombinant cell comprising the second selectable marker and the nucleic acid of interest.

19. A method for generating a recombinant Cyanobacterium cell comprising a first nucleic acid of interest, the method comprising:
(a) contacting the cell of claim 16 with a first nucleic acid insert under conditions that allow recombination of the first nucleic acid insert with the nucleic acid cassette in the genome of the cell; wherein
the first nucleic acid insert comprises, in a 5′ to 3′ direction, the first landing zone, the first nucleic acid of interest, a fifth landing zone, a second selectable marker, and the third landing zone, or wherein the first and/or third landing zones have at least 90% sequence identity to the first and/or third landing zones in the cell, respectively; and
(b) culturing the cell in the presence of a selection agent for the second selectable marker, thereby selecting a recombinant cell comprising the second selectable marker and the first nucleic acid of interest.

20. The method of claim 19 further comprising
(c) contacting a cell from step (b) with a second nucleic acid insert under conditions that allow recombination of a second nucleic acid insert with the nucleic acid construct in the genome of the cell; wherein
the second nucleic acid insert comprises, in a 5′ to 3′ direction, the fifth landing zone, a further selectable marker, a sixth landing zone, a second nucleic acid of interest, and the fourth landing zone, or wherein the fourth and/or fifth landing zones have at least 90% sequence identity to the fourth and/or fifth landing zones in the cell, respectively; and
(d) culturing the cell in the presence of a selection agent for the further selectable marker, thereby selecting a recombinant cell comprising the further selectable marker, the first nucleic acid of interest and the second nucleic acid of interest.

21. A method for generating a recombinant Cyanobacterium cell comprising a first nucleic acid of interest, the method comprising:
(a) contacting the cell of claim 16 with the first nucleic acid insert under conditions that allow recombination of the first nucleic acid insert with the nucleic acid construct in the genome of the cell; wherein the first nucleic acid insert comprises, in a 5′ to 3′ direction, the second landing zone, a second selectable marker, a sixth landing zone, the first nucleic acid of interest, and the fourth landing zone, or wherein the second and/or fourth landing zones have at least 90% sequence identity to the second and/or fourth landing zones in the cell, respectively;
(b) culturing the cell in the presence of a selection agent for the second selectable marker, thereby selecting a recombinant cell comprising the second selectable marker and the first nucleic acid of interest.

22. The method of claim 21, further comprising
(c) contacting a cell from step (b) with a second nucleic acid insert under conditions that allow recombination of the second nucleic acid insert with the nucleic acid construct in the genome of the cell; wherein the second nucleic acid insert comprises, in a 5′ to 3′ direction, the first landing zone, a second nucleic acid of interest, the fifth landing zone, a further selectable marker, and the sixth landing zone, or wherein the first and/or sixth landing zones have at least 90% sequence identity to the first and/or sixth landing zones in the cell, respectively; and
(d) culturing the cell in the presence of a selection agent for the further selectable marker, thereby selecting a recombinant cell comprising the further selectable marker, the first nucleic acid of interest and the second nucleic acid of interest.

23. The method of claim 17, wherein the one or more of the nucleic acid of interest, first nucleic acid of interest, or second nucleic acid of interest is operatively coupled to a constitutive or inducible promoter.

24. (canceled)

25. (canceled)
